(19) United States
(12) Patent Application Publication
(10) Pub. No.: US 2018/0071190 A1
(43) Pub. Date: Mar. 15, 2018

(54) COSMETIC PRODUCT AND CONCENTRATE FOR PRODUCING THE COSMETIC PRODUCT

(71) Applicant: GCT GMBH, Leichlingen (DE)

(72) Inventors: Martin ALBRECHT, Odenthal (DE); Bernd KOMP, The Hague (NL)

(21) Appl. No.: 15/560,356

(22) PCT Filed: Mar. 10, 2016
(86) PCT No.: PCT/DE2016/000107

§ 371 Date: Sep. 21, 2017

(30) Foreign Application Priority Data
Mar. 25, 2015 (DE) 10 2015 003 841.9

(51) Int. Cl. A61K 8/55; A61K 8/34; A61K 8/92; A61K 8/02; A61Q 19/00

(52) U.S. Cl. CPC A61K 8/553; A61K 8/345; A61Q 19/00; A61K 8/0295; A61K 8/922

(57) ABSTRACT

A cosmetic product is described which in addition to water, contains at least one hydrogenated phospholipid in a concentration of at least 0.7% by weight, at least one divalent and/or trivalent alcohol, and at least one wax. In the cosmetic product, the weight ratio of hydrogenated phospholipid to the wax varies between 1:0.3 and 1:1.5, more preferably between 1:0.7 and 1:1, wherein the hydrogenated phospholipid is at least partially present in an ortho-rhombic lamellar crystalline structure and the wax is incorporated in the ortho-rhombic lamellar crystalline structure and/or is attached to the ortho-rhombic lamellar crystalline structure.

### FIELD OF THE INVENTION

The present invention relates to a cosmetic product which contains, in addition to water, at least one hydrogenated phospholipid in a concentration of at least 0.7% by weight, at least one divalent and/or trivalent alcohol and at least one wax and a concentrate for manufacturing such a cosmetic product.

### BACKGROUND OF THE INVENTION

A cosmetic product having the features of the preamble of patent claim 1 is known from WO 2009/043341. The known composition here contains, as ingredients, a hydrogenated phospholipid in a concentration of at least 0.7% by weight, at least one divalent and/or trivalent alcohol, and at least one wax, wherein only in the embodiment M, the concentration of the wax, which is rice bran wax, is quantified with 0.1% by weight, whereas in this exemplary embodiment, the concentration of the hydrogenated phosphatidylcholine is specified as 1.5% by weight. From this, a ratio of hydrogenated phospholipid to wax is calculated to be 1:0.066. Mainly, however, WO 2009/043341 discusses the known formulation having lamellar structures, which comprise sandwich-like lamellar double membrane layers arranged one above the other. A layer of an internal aqueous phase is respectively provided between adjacent double membrane layers arranged parallel to one another. The active ingredients, irrespective of whether they are lipophilic or hydrophilic, are in each case arranged in the double membranes and in the internal aqueous phases, but in different concentrations, while the outer aqueous phase which surrounds the lamellar structure is largely free of active ingredients.

### SUMMARY OF THE INVENTION

The object of the present invention is to provide a cosmetic product of the type specified which has a particularly high activity with respect to the prevention or elimination of barrier disorders of the skin.

Furthermore, the present invention is based on the object of providing a concentrate from which the cosmetic product can be prepared particularly simply and quickly by diluting with an aqueous system.

These objects are achieved according to the invention by a cosmetic product, wherein a) in the cosmetic product, a weight ratio of hydrogenated phospholipid to the wax varies between 1:0.3 and 1:1.5, in particular between 1:0.7 and 1:1, b) in the cosmetic product, the hydrogenated phospholipid is at least partially present in an ortho-rhombic lamellar crystalline structure, and c) the wax is incorporated in the ortho-rhombic lamellar crystalline structure and/or is attached to the ortho-rhombic lamellar crystalline structure and by a concentrate, wherein a) the concentrate is prepared by dilution with an aqueous system, b) wherein the concentrate contains at least one hydrogenated phospholipid, water, at least one dihydric and/or trihydric alcohol and at least one vegetable wax, c) wherein the concentrate can be diluted with the aqueous system with the formation of the cosmetic product in a volume ratio between 1:0.3 and 1:15, in particular between 1:0.7 and 1:1, d) the concentrate has such a concentration of hydrogenated phospholipid that, depending on the desired dilution, the cosmetic product produced therefrom by dilution contains at least 0.7% by weight of the hydrogenated phospholipid, e) wherein in the concentrate, the weight ratio of hydrogenated phospholipid to the vegetable wax varies between 1:0.3 and 1:1.5, in particular between 1:0.7 and 1:1, f) wherein in the concentrate, the hydrogenated phospholipid is at least partially present in an ortho-rhombic lamellar structure, and g) wherein in the concentrate, the wax is incorporated in the ortho-rhombic lamellar crystalline structure and/or is attached to the ortho-rhombic lamellar crystalline structure.

According to the invention, therefore, a cosmetic product is proposed which, in addition to water, the above-described formulation contains at least one hydrogenated phospholipid in a concentration of at least 0.7% by weight, at least one divalent and/or trivalent alcohol, and at least one wax. In contrast to the known formulation described above, however, in the cosmetic product according to the invention, the weight ratio of hydrogenated phospholipid to the wax is quantified to the effect, that in the cosmetic product according to the invention, the weight ratio of hydrogenated phospholipid to wax varies between 1:03 and 1:1.5, in particular between 1:0.7 and 1:1.1. Furthermore, in the cosmetic product according to the invention, the hydrogenated phospholipid is at least partially present in an ortho-rhombic lamellar crystalline structure, wherein this ortho-rhombic lamellar crystalline structure is explained in detail below. In the ortho-rhombic lamellar crystalline structure, the wax is incorporated and/or attached to the ortho-rhombic lamellar crystalline structure.

As the inventors of the present application have surprisingly determined, the previously described known composition also has a lamellar structure, but this lamellar structure is a hexagonal lamellar structure and not, as proposed according to the invention, an ortho-rhombic lamellar crystalline structure. Furthermore, it is regarded as essential according to the invention that the desired ortho-rhombic lamellar crystalline structure contained in the cosmetic product according to the invention is formed only when the weight ratio of hydrogenated phospholipid to the wax varies between 1:0.3 and 1:1.5, in particular between 1:0.7 and 1:1.1. If this weight ratio is fallen below or exceeded, liquid lamellar structures or hexagonal lamellar structures are then formed from the hydrogenated phospholipid and the wax incorporated therein and/or attached thereto, but no ortho-rhombic lamellar crystalline structures therefrom, as is described and discussed in detail below in the exemplary examples.

It is precisely these ortho-rhombic lamellar crystalline structures, which are formed exclusively at the previously quantified weight ratios of hydrogenated phospholipid to the wax, which are able to prevent barrier disorders of the skin and furthermore to eliminate barrier disorders of the skin particularly effectively so that the cosmetic product according to the invention can be excellently used not only prophylactically but also therapeutically for the treatment of barrier disorders of the skin, as is subsequently proved based on the efficacy studies described in the exemplary examples.

The above-described improved prophylactic and therapeutic cosmetic efficacy of the cosmetic product according to the invention leads the inventors back to the fact that the cosmetic product according to the invention particularly effectively prevents or eliminates the occurrence of imperfections or the presence of defects in the intercellular lipids of the callused skin whereby the transepidermal water loss, which is a measure of the above-mentioned barrier disorders, is held in a range or is returned to a range which corresponds to the healthy, non-damaged skin. Due to the stiffer, more compact structure of the ortho-rhombic lamellar crystalline structure, which has a low mobility of the molecules and a more compact structure as compared to a hexagonal lamellar structure, the cosmetic product according to the invention is essentially better able to eliminate or prevent the above-mentioned barrier disorders and particularly defects in the intercellular lipids better, faster and more effectively than a hexagonal lamellar structure or a liquid lamellar structure. The product according to the invention also does not penetrate so deeply into the lower layers of the skin, but rather remains mainly in the callus layer (horny layer) and ensures here that the intercellular lipids which exist between the horny multi-layered squamous epithelium form no or fewer defects or the defects present there are repaired by the ortho-rhombic lamellar crystalline structure.

As a hydrogenated phospholipid which is contained in the cosmetic product according to the invention, basically, any hydrogenated phospholipid is suitable which builds ortho-rhombic lamellar crystalline structures with the wax in the above-described weight ratios. In particular, in the cosmetic product of the present invention, such hydrogenated phospholipids are provided which are selected from the group consisting of hydrogenated phosphatidylethanolamine, hydrogenated phosphatidylinositol, hydrogenated phosphatidylcholine, hydrogenated lyso-phosphatidylcholine, hydrogenated phosphatidylserine, and hydrogenated phosphatidic acid.

Particularly good results with regard to the prevention and/or the elimination of barrier disorders and, in particular, of defects in the intercellular lipids and thus to the maintenance of a healthy skin and/or the treatment of damaged skin have such embodiments of the cosmetic product according to the invention in which the hydrogenated phospholipid consists of vegetable phospholipids, preferably from soy lecithin or sunflower lecithin, is produced by hydrogenation and has a concentration of hydrogenated phosphatidylcholine of at least 60% by weight, based on the hydrogenated phospholipid used. The prophylactic and therapeutic effectiveness of the cosmetic product according to the invention is further improved by using a hydrogenated phospholipid having a concentration of hydrogenated phosphatidylcholine of between 70% by weight and 85% by weight and in particular between 90% by weight and 98% by weight.

With respect to the wax which is contained in the cosmetic product according to the invention and which forms the ortho-rhombic lamellar crystalline structure with the hydrogenated phospholipid in the above-mentioned weight ratios, a vegetable wax that is isolated from leaves, needles, stalks, roots, rinds, bran, peels, seeds, flowers and/or fruits is particularly selected. These include, in particular, such vegetable waxes (alone or in mixture) which are selected from the group consisting of carnauba wax, candelilla wax, ouricuri wax, sugar cane wax, retamow wax, caranday wax, raffia wax, columbine wax, esparto wax, alfalfa wax, bamboo wax, hemp wax, douglas fir wax, coconut wax, sisal wax, flax wax, cotton wax, damen wax, flax wax, cotton wax, dammar wax, cereal wax, tea wax, coffee wax, ocatilla wax, citrus aurantium dulcis peel wax, Ficus Ceriferous wax, orange wax, sunflower seed wax, sunflower seed shell wax, sprout kale wax, tobacco plant wax, pumpkin seed wax, corn wax, prickly cactus wax and oleander wax.

A particularly suitable highly effective embodiment of the cosmetic product according to the invention has in particular the carnauba wax, sunflower seed wax, rice wax or the rice bran wax alone or in a mixture with one another or in a mixture with the previously described waxes. Particularly when the wax, and preferably the carnauba wax, sunflower seed wax, rice wax and/or rice bran wax, which are chemically regarded as esters, contain saturated C₂₂-C₂₆ fatty acids as a main fatty acid component, the advantages spoken of at the onset of the cosmetic product according to the invention are achieved in particular to a high degree, wherein it is particularly preferred when the wax used, and preferably the carnauba wax, sunflower seed wax, rice wax and/or rice bran wax, contains this main fatty acid component in a concentration of between 15% by weight and 33% by weight, in particular between 23% by weight and 28% by weight, based on the weight of the respective wax.

Another particularly preferred embodiment of the cosmetic product according to the invention proposes here that the wax and preferably the carnauba wax, sunflower seed wax, rice wax or the rice bran wax additionally contains free linear C₃₀-C₃₄ alcohols and/or free C₁₆-C₂₄ fatty acids, in particular in a fatty acid concentration between 1% by weight and 16% by weight, based on the weight of the respective wax.

In order to improve the storage stability of the cosmetic product according to the invention, it is particularly advantageous to limit the concentration of the unsaturated fatty acids in the above-described free C₁₆-C₂₄ fatty acids, wherein in particular the concentration of the unsaturated fatty acids in the free fatty acids is or is set between 0.05% by weight and 0.4% by weight, based on the weight of the particular wax or the wax mixture, and in particular based on the weight of the carnauba wax, sunflower seed wax, rice wax and/or the rice bran wax.

Furthermore, another embodiment of the cosmetic product according to the invention provides for the extension of the storage stability and for the prevention of undesirable oxidation in that the cosmetic product according to the invention here has at least one antioxidant. Preferably, this antioxidant is selected from the group consisting of tocopherols, polyphenols, epigallocatechins, epigallocatechin gallate, caffeic acid, flavonoids, ellaginic acid, curcum in derivatives, dihydroquercetin, tetrahydrocurcuminoid, tetrahydrodiperuloylmethanes, L-carnosine, N-acetylcysteine, phytic acid, chelating agents, in particular thioctic acid and/or EDTA, BHA, BHT, vegetable ingredients, such as Picea abies extract, pyogenogenol, Bakuchiol, hydroxityrosol, and derivatives, bis-ethylhexyl hydroxydimethoxy benzylmalonate (Ronacare AP, manufacturer Merck) lipochromans, especially lipochroman-6. Depending on the particular antioxidant, the concentration thereof preferably varies between 0.01% by weight and 10% by weight, based on the ready-for-use cosmetic product.

As already described above in the case of the cosmetic product according to the invention, the cosmetic product contains a divalent and/or trivalent alcohol, wherein it is preferably a diol and/or glycerin. Particularly preferred alcohols are, in addition to glycerol, pentylene glycol, in particular 1,2-pentanediol, hexylene glycol, in particular 1,2-hexanediol, octanediol and/or butylcyclohexanol, preferably tertiary butylcyclohexanol and in particular 4-t-butylcyclohexanol, in each case alone or in any desired mixture. In particular, the above-mentioned alcohols have a particularly high skin compatibility and promote the formation of the ortho-rhombic lamellar crystalline structure.

A particularly preferred embodiment of the cosmetic product according to the invention provides that the cosmetic product contains isostearyl isostearate as a further ingredient in a concentration of between 1% by weight and 20% by weight. In particular, this provides that the cosmetic product has a skin-protecting property.

The butylcyclohexanol already described in the alcohols, and in particular the tertiary butylcyclohexanols, and preferably the 4-t-butylcyclohexanol, are not only preferred alcohols, but also excellent active ingredients which belong to the group of TRPV1 inhibitors (TRPV1_=transient receptor potential channel, vanilloid subfamily member 1), which lower the irritation threshold of the skin and counteract skin reactions, which, among other things, occur in sensitive, barrier-disorder skin condition.

Regarding the concentration of hydrogenated phosphatidylcholine in the cosmetic product according to the invention, it should be noted that this concentration varies in particular between 0.7% by weight and 8% by weight, preferably between 1.2% by weight and 5% by weight.

The cosmetic product according to the invention further has a light protection filter, a UV filter, a skin-protecting active ingredient, a skin-care active ingredient, a smoothing active ingredient, a skin-softening active ingredient, a skin-whitening active ingredient, a tanning active ingredient, a deodorizing active ingredient, a depilatory active ingredient, a moisturizing active ingredient, an active ingredient for the care and treatment of hypersensitive skin, an active ingredient for the treatment and care of infected, irritated or diseased skin, an active ingredient for prophylaxis against insect bites, a greasing active ingredient, an anti-inflammatory active ingredient and/or a moisturizing active ingredient.

Preferred light protection filters and UV filters are in particular benzophenone-3, benzophenone-4, benzophenone-5,3-benzylidene camphor, benzylidenecamphor sulfonic acid, butyl methoxydibenzoylmethane, camphor benzalkonium methosulfate, diethylhexyl butamido triazone, ethylhexyldimethyl PABA, ethylhexyl methixycinnamate, ethylhexyl salicylate, ethylhexyl triazone, homoalate, isoamyl p-methoxycinnamate, 4-methylbenzylidene camphor, octocrylene, PABA (p-aminobenzoic acid), PEG-25 PABA, phenylbenzimidazole sulfonic acid, polyacrylamidomethyl benzylidene camphor, potassium phenylbenzimidazole, sodium phenylbenzimidazole sulfonate, TEA-phenylbenzimidazole sulfonates and terephthalylidene dicamphor sulfonic acid, oxybenzene, BEMT, octocrylene, benzophenone-9, diethylam ino-hydroxybenzoylhexylbenzoate, drometrizoles trisiloxane, 4-methylbenzylidene camphor, 3-benzylidenes camphor, octylsalicylate, methylene bis-benzotriazolyl tetramethylbutylphenol, bis-ethylhexyloxyphenol methoxyphenyl triazine, ethylhexylmethoxycinnamates, diethylhexyl butamido triazones, phenylbenzimidazole sulfonic acid, diethylamino hydroxybenzoyl hexyl benzoates, disodium phenyl dibenzimidazoles tetrasulfonates, titanium dioxide, zinc oxide and terephthalylidenes dicamphor sulfonic acid.

Depending on the respective light protection filter or UV filter, its concentration in the cosmetic product according to the invention is between 5% by weight and 30% by weight, in particular between 10% by weight and 20% by weight, based in each case on the weight of the ready-to-use cosmetic product.

In another embodiment, the cosmetic product according to the invention has allantoin, chlorogenic acids, colostrum, lactobacillus/algal ferment, laminaria digitata extract, laminaria japonica extract, mimosa tenuiflora bark extract, Plantago ovata extract, polygonum fagopyrum extract, potassium ascorbyl tocopheryl phosphate, PVP/eicosencopolymer, PVP/hexadecancopolymer, salvia officinalis extract, sophora japonica extract, sphingolipids, spirulina platensis extract, tocopherylinoleate, vitis vinifera seed extract and/or yeast betaglucan, the preferred concentration of which varies in particular between 0.005% by weight and 40% by weight, more preferably between 0.005% by weight and 10% by weight.

The preferred skin-care active ingredients which are optionally present individually or as a mixture in the cosmetic product according to the invention are in particular actelyglucosamine, adenosine, cyclic adenosine phosphate, adenosine phosphate, adenosine triphisohate, alchemilla vulgaris extract, ammi visnaga extract, ammonium glycyrrhizinate, anthemis nobilis extract, arbutine, arctium lappa extract, Asian acid, Aspergillus ferment, atelocollagenatela sativa protein, beta carotene, betaglucan, beta-sitosterol, biosaccharides gum-1, biotin, bombyxe extract, butyrospermum parkii butter, C12-20 isoparaffin, C14-18 glycol, C16-36 alkyl stearate, C18-30 glycol, C20-24 olefin, C20-30 glycol, C24 -28 olefin, C30-45 alkyl dimethicone, C30-50 alkyl bee wax, C30-50 alkyl stearate, C40-60 alkyl stearate, ceramide 1, ceramide 1A, ceramide 2, ceramide 4, ceramide 5, ceramide 611, ceramide 3, DI-C12-13 alkyltartrate, echinacea agustifolia extract, elastin, elastin amino acids, folic acid, glucuronolactone, glutamine, glyceryl stearate citrate, glycine, glycine soybean extract, glycine soybean flower, glycine soybean seed extract, glycine soybean oil, glycine soybean oil (unsaponifiable), glycine soybean protein, glycyrrhetic acid, glycyrrhetinyl stearate, glycyrrhizinic acid, guar hydroxypropylthimonium chloride, Harpagophytum procumbens extract, Helianthus annuus extract, Helianthus annuus seed extract, Helianthus annuus seed oil, heliotropin, hexamethyldisiloxane, hexamidine diisethionate, hippophae rhamnoides oil, hordeum vulgare extract, hydrogenated cottonseed glyceride, hydrogenated cottonseed oil, hydrogenated jojoba oil, hydrogenated jojoba wax, hydrogenated lanolin alcohol, hydrogenated palm glyceride, hydrogenated palm glyceride citrate, hydrogenated palm kernel glyceride, hydrogenated palm oil, hydrogenated palm/ palm kernel oil PEG-6 ester, hydrogenated peanut oil, hydrogenated polyisobutene, hydrogenated rapeseed oil, hydrated plant glycerides, hydrogenated vegetable oil, hydrolyzed protein, hydrolyzed casein, hydrolyzed collagen, hydrolyzed corn protein, hydrolyzed corn starch, hydrolyzed DNA, hydrolyzed egg shell membrane, hydrolyzed elastin, hydrolyzed extensin, hydrolyzed fibronectin, hydrolyzed gadidae protein, hydrolyzed gelatin, hydrolyzed glycosaminoglycans, hydrolyzed glycyrrhizinate, hydrolyzed hair keratin, hydrolyzed milk protein, hydrolyzed silk, hydrolyzed soybean, hydrolyzed soy protein, hydrolyzed soybean starch, hydrolyzed vegetable protein, hydrolyzed wheat gluten, hydrolyzed wheat protein, hydrolyzed wheat protein/dimethiconcopoliolphosphat copolymer, hydrolyzed wheat starch, hydrolyzed yeast, hydrolyzed yeast protein, hydrolyzed zein, hyroxylauroylphytosphingosin, hydroxyproline, hydroxypropylcyclodextrin, hydroxypropyltrimonium, hydrolyzed rice bran protein, hydroxypropyltrimonium hydrolyzed silk, hydroxypropyltrimonium hydrolyzed soy protein, hydroxypropyltrimonium hydrolyzed vegetable protein, isoamyllaurate, isobutyl myristate, isobutyl palm itate, isobutyl pelargonate, isobutyl stearate, isobutyl tallowate, isocetyl behenate, isocetyl isostearate, isodecyl neopentanoate, isohexadecane, isohexyl laurate, isohexyl neopentanoate, isohexyl palm itate, isolauryl behenate, isopropyl isostearate, isostearyl isostearate, jojoba alcohol, jojoba ester, keratin, keratin amino acids, lactic acid, lactobacillus ferment, lactoferrin, lactoglobulin, lactose, lactoyl phytosphingosine, lanolin, lanolin cera, lanosterol, lauramidopropylbetaine, lauryldimonium hydroxypropyl hydrolyzed soy protein, lauryldimonium hydroxypropyl hydrolyzed wheat protein, lecithin, linoleic acid, linolenic acid, linum usitatissimum extract, linum usitatissimum oil, lonicera japonica extract, lysine, lysinapartate, macadamia ternifolia extract, magnesium ascorbate, magnesium gluconate, magnesium PCA, maltodextrin, marrubium vulgare extract, Medicago sativa oil (unsaponifiable) melia azadirachta extract, methyl laurate, methyllinoleate, methyl myristate, methyl silanol mannuronate, milk amino acids, moringa seed oil, meadowscale seed oil, moringa butter myristyl lactate, myristyl myristate, myristyl neopentanoate, oenothera biennis extract, olea europaea extract, Ophiopogon japonicus extract, Oryza sativa cera, Oryza sativa extract, Oryza sativa cera, oryzanol, panthenol, pantothenic acid, papain, PCA dimethicone, PEA palmitate, phytosphingosine, polyquaternium 47, potassium aspartate, potassium cocoyl hydrolyzed grain protein, potassium glycyrrhizinate, procollagen, proline, protease, PTFE, pyridinedicarboxylic acid, pyridoxal-5 phosphate, pyridoxine-HCI, pyrus malus extract, quaternium-79 hydrolyzed silk, quaternium-79 hydrolyzed soy protein, quaternium-79 hydrolyzed wheat protein, reseda luteola extract, resorcinol acetate, retinol, retinyl acetate, retinyllinoleate, retinyl palmitate, retinylpropionate, ribes nigrum extract, riboflavin, royal jelly, royal jelly extract, ruscogenin, Ruscus aculeatus extract, Ruta graveolens extract, rutin, Saccharomyces ferment, Saccharomyces lysate extract, Salix alba rind extract, salvia officinalis, salvia officinalis extract, sericin, serine, serum albumin, serum protein, shellac wax, silk amino acids, silicium lupids, silybum marianum extract, simethicone, Simmondsia chinensis wax, Simmondsia chinensis extract, sodium ascorbyl/cholesteryl phosphate, sodium hyaluronate crosspolymer, sodium hyaluronate dimethylsilanol, sodium isethionate, sodium lauroyl isethionate, sodium lauroyl oataminic acids, sodium lauroyl sarcosinate, sodium lauroyl silk amino acids, sodiumlevulinate, sodium PCA, sodium PCA methylsilanol, sodium polyglutamate, sorbitol, soyasterol, sphingolipids, squalane, stearamidopropylbetaine, stearylglycyrrhetinate, stearylactate, sucrose, sunflower seed oil glycerides, symphytum officinale leaf extract, theobroma cacao extract, tocopherylolate, Triticum vulgare extract, troxerutin, tyrosine, ubichonon, undecyl alcohol, urea, vegetable glyceride phosphates, verbena officinalis extract, vitamins and their derivatives, preferably vitamin A and E, wheat amino acids, zea mays, zinc aspartate, zinc DNA, zinc glucoheptonate, zinc gluconate, zinc glutamate, zinc hydrolized collagen and/or zinc PCA.

Depending on the respective active substance and the intended use, the preferred concentration of the skin-care active substances listed above varies between 0.005% by weight and 40% by weight.

For the smoothing or wrinkle-reducing active ingredients, Boswelia serrata extract, Centella asiatica extract, glycyrrhiza glabra, glycyrrhiza glabra extract, morus alba extract, niacin, niacinamide, persea gratissima extract, sericine, pentapeptides, hexapeptides, in particular hexa peptides-2 and/or hexapeptides-9, heptapeptides, copper peptides, growth factors of the TGF beta family, MPC milk peptides, MTP milk tripeptides, palmitoyl oligopeptides/matrices, in particular Pal KTTKS (manufacturer: Sederma) and/or Pal-VGVAPG (manufacturer: Sederma), acetylhexapeptides 3, palm itoylpentapeptides, palm itoyltripeptides-5, serilesine (=laminin, manufacturer: Lipotec), lipeptide (=oligopeptide, manufacturer: Lipotec), tripeptide 10-citrulline, Aldenine (manufacturer: Lipotec), polygamma-glutamine acid, alanine, serine, glycine, arginine, glutamic acid, histidine and spirulina maxima extract are included in the cosmetic product according to the invention in a preferred concentration between 0.001% and 40% by weight.

The group of softening active ingredients includes in particular arachis hypogaea oil, arctium lappa seed oil, avena sativa extract, behenyl alcohol, borago officinalis extract, borago offincinalis seed oil, brassica campestris oleifera oil, butyrospermum parkii butter, butyrospermum parkii butter extract, butyrospermum parkii butter (unsaponifiable) , calendula officinalis extract, calendula officinalis oil, candelilla cera, canola oil, caprylyl glycol, carthamus tinctorius extract, carthamus tinctorius oil, cera alba, cera carnauba, ceratonia siliqua extract, cetearyl alcohol, cetearyl isononanoate, cetyl alcohol, cetyl ethylhexanoate, cetyl palmitate, cetyl stearate, cholesterol, cholesteryl hydroxystearate, cholesteryl isostearate, cholesteryl macadamate, cholesteryl nonanoate, cholesteryl stearate, cocoglycerides, cocos nucifera extract, cocos nucifera oil, cocoyl glutamic acid, coenzyme A, coriandrum sativum seed oil, cyclohexasiloxane, cyclomethicone, cyclopentasiloxane, cyclotetrasiloxane, cyclo trisiloxane, dibutyl adipate, dibutyl sebacate, diethyl sebacate, diethyl succinate, dihydrocholesterol, dihydrocholesteryl butyrate, dihydrocholesteryl isostearate, dihydrocholesteryl macadamate, dihydrocholesteryl nonanoate, dihydrocholesteryl octyl decanoate, dihydrocholesteryl oleate, diisobutyl adipate, equisetum arvense extract, erucyl arachidate, erucyl erucate, ethyl olivate, glyceryl caprate, glyceryl caprylate, glyceryl caprylate/caprate, glyceryl cocoate, glyceryl diarachidate, glyceryl dibehenate, glyceryl dierucate, glyceryl dihydroxystearate, glyceryl diisopalmitate, glyceryl diisostearate, glyceryl dilaurate, glyceryl dilinoleate, glyceryl dimyristate, glyceryl dioleate, glyceryl dipalmitate, glyceryl dipalmitoleate, glyceryl diricinoleate, glyceryl distearate, glyceryl erucate, glyceryl ethylhexanoate/stearate/adipate, hydrogenated glyceryl soyate, glyceryl hydroxystearate, glyceryl isostearate, glyceryl isostearate/myristate, glyceryl isostearates, glyceryl lanolate, glyceryl laurate, glyceryl laurate/oleate, glyceryl linoleate, glyceryl myristate, glyceryl oleate, glyceryl oleate/elaidate, glyceryl palmitate, glyceryl palmitate lactate, glyceryl palmitate/stearate, glyceryl ricinoleate, glyceryl ricinoleate SE, glyceryl sesquioleate, glyceryl stearate, glyceryl stearate citrate, glyceryl stearate diacetate, glyceryl stearate lactate, glyceryl stearate succinate, glyceryl stearate/acetate, glyceryl stearate/maleate, glyceryl tallowate, glyceryl triacetyl hydroxystearate, glyceryl triacetyl ricinoleate, glyceryl/sorbitol oleate/hydroxystearate, glycosphingolipids, glycyrrhiza glabra, glycirrhiza glabra extract, cotton oil, helianthus annuus extract, helianthus annuus seed oil, hexamidine diisethionate, hippophae rhamnoides oil, hordeum vulgare extract, humulus lupulus extract, hydrogenated coco glycerides, hydrogenated jojoba oil, hydrogenated jojoba wax, hydrogenated lanolin, hydrogenated palm glycerides, hydrogenated palm kernel oil, hydrogenated polydecene, hydrogenated rapeseed oil, hydrogenated vegetable oil, hydrolyzed collagen, Hypericum Perforatum oil, isohexadecane, isopropyl isostearate, lauryldimonium hydroxypropyl collagen Limnanthes albacea oil, macadamia ternifolia extract, palmitoyl collagen (hydrolyzed), panax ginseng extract, paraffin, paraffin liquidum, rices nigrum extract, ribes nigrum oil, simmondsia chinensis cera, simmondsia chinensis extract, simmondsia chinensis oil, solanum lycopersicum oil, sorbitan distearate, candelilla wax (synthetic), carnauba (synthetic), japan wax (synthetic), jojoba oil (synthetic), wax (synthetic), triethylhexyl citrate and verbena officinalis extract. The preferred concentration of these softening active ingredients is between 0.5% by weight and 40% by weight.

The group of the skin-whitening active ingredients which can be present in the cosmetic product according to the invention includes in particular magnesium ascorbyl phosphate, di-sodium ascorbyl phosphate, tetrahydrodiferuloyl-methane, lepidium sativum sprout extract, hydroquinone, tretinoin, azelainic acid, koji acid, kojiic dipalmitate, arbutin, bayberry extract, paper mulberry extract, glabridine, licorice extract, ascorbic acid, glyxyrrhiza uralensis extract, melanostat, octadecendioc acid, phenylpropanoids, zinc glycine complexes, tretionine, waltheria indica leaf extract, hydroxyphenoxy propionic acid, undecylenoyl phenylalanine, ascorbyl tetraisopalmitate, mandresey extract, ascorbic acid 2-glucoside and 441-phenylethyl)1,3-benzenediol. Depending on the particular active ingredient, its concentration preferably varies between 0.01% by weight and 7% by weight.

As tanning active ingredients, acetyltyrosine, erythtulose and dihydroxyacetone are preferably present in a preferred concentration of between 0.1% by weight and 10% by weight in the cosmetic product according to the invention.

For the deodorizing active ingredients, aluminum chlorohydrate, triethyl citrates, silver citrates, sodium caproyl/lauroyl lactylates in a preferred concentration of between 0.1% and 15% by weight are to be mentioned in the cosmetic product according to the invention.

The group of depilatory active ingredients includes, in particular, ammonium thiolactate, calcium thioglycolate, mercaptopropionic acid, potassium thioglycolate, sodium thioglycolate and mercaptopropionic acid in a preferred concentration of between 5% by weight and 10% by weight.

Moisturization active ingredients are preferably arginine PCA, butylene glycol, butyloctanol, calcium gluconate, carboxymethyl-chitosan succinamide, chitosa PCA, copper-acetyl-tyrosinate-methylsilanol, copper PCA, copper-PCA-methylsilanol, serine, glycine, alanine, sodium polyaspertate, betaine, urea, dipotassium glycyrrhizate, erythritol, ethoxydiglycol, ethylhexyl-PCA, galactonolactone, glucamine, glutamic acid, glycyrrhizic acid, hyaluronic acid, inositol-hexa-PCA, isomalt, lysine PCA, magnesium PCA, maltitol, phytantriol, potassium PCA, saccharide hydrolyzate, sodium carboxymethyl chitin, sodium hyaluronate, sodium hyaluronate crosspolymer, sodium hyaluronate dimethylsilanol, sodium lactate, sodium PCA, sodium polyaspartate, sodium polyglutamate, sorbitol, soyasterol, urea, xylitol, xylose, fillagrin stimulating substances, such as madecassosides, wheat proteins, alteromonas ferment filtrates, hydrolyzed beta glucan (hydreis, impag), revidrate (sederma), PCA (pyrrolid carboxylic acid) and derivatives in a preferred concentration of between 0.05% and 25% by weight, based on the weight of the ready-to-use cosmetic product.

In particular, the active ingredients to be mentioned are butylcyclohexanol, preferably butylcyclohexanols and, in particular, 4-t-butylcyclohexanol and palmitoyl tripeptides-8, which are present in a preferred concentration between 0.01% by weight and 2.5% by weight, for the care or treatment of hypersensitive skin, in the cosmetic product according to the invention.

A particularly high cosmetic effectiveness with regard to the maintenance of a healthy skin barrier and with respect to the restoration of a damaged skin barrier is achieved in a further development of the cosmetic product according to the invention by the product according to the invention having a mixture of butylcyclohexanol, in particular from tertiary butylcyclohexanols and preferably from 4-t-butylcyclohexanol with propylene glycol and/or pentylene glycol.

In order to treat with the cosmetic product according to the invention in particular infected, irritated or diseased skin regions, a further development provides that the cosmetic product according to the invention contains an anti-inflammatory active substance which is selected from the group consisting of ursolic acid, soya sterol, 18-beta-glycyrrhetinic acid, gamma-oryzanol, ferula acid, avenanthramide, boswellic acid, asciaticoside, magcassoside, CM glucan, troxerutin, rutin, monoalkanolamides, rosmarinic acid, marigold extract, St. John's wort extract, Cardiospermum halicacabum extract, camomile extract, sunhute extract and derivatives of the aforementioned anti-inflammatory or anti-itching active ingredients, wherein the preferred concentration is between 0.01% by weight and 5% by weight.

In order to achieve prophylaxis against insect bites in the cosmetic product according to the invention, this embodiment of the product according to the invention comprises an active ingredient selected from the group consisting of iscaridine, clove oil, citronellal, cedarwood oil, lavender oil, cinnamon oil, permethrin and crotamiton. In particular, these active substances prevent the user from being protected in particular by stings of mosquitoes, fleas, lice and/or ticks, wherein the preferred concentration varies between 0.1% by weight and 10% by weight.

The cosmetic product according to the invention preferably has, as a greasing active ingredient, wheat germ glycerides in a preferred concentration of between 0.1% by weight and 5% by weight.

The cosmetic product according to the invention in particular contains as moisturizing active ingredients dimethylsilanol-hyaluronate, glycine-soy extract, glycyrrhiza glabra and/or manganese PCA in a preferred concentration between 0.05% and 5% by weight.

A further embodiment of the cosmetic product according to the invention, which is used, in particular, for the care and treatment of skin which is sensitive to noxa, and against neurogenically induced skin inflammations, has, as active ingredients, butylcyclohexanol, resolvin, farnesyl pyrophosphates, capsazepines, cinnamide, carboxamide and/or palmitoyl tripeptide-8, wherein the preferred concentration of these active ingredients is between 0.01% by weight and 1% by weight.

The present invention further relates to a concentrate for manufacturing the above-described cosmetic product according to the invention, wherein the concentrate is preparable by dilution with an aqueous system, and the concentrate contains at least one hydrogenated phospholipid, water, at least one dihydric and/or trivalent alcohol and at least one vegetable wax. According to the invention, it is proposed that the concentrate can be diluted with the aqueous system in a volume ratio between 1:0.3 and 1:15 with the formation of the cosmetic product, wherein the concentrate has a concentration of hydrogenated phospholipid such that, depending on the desired dilution, the cosmetic product produced by dilution contains at least 0.7% by weight of the hydrogenated phospholipid. Furthermore, in the concentrate according to the invention, the weight ratio of hydrogenated phospholipid to the vegetable wax varies between 1:0.3 and 1:1.5, in particular between 1:0.7 and 1:1.1. In the concentrate according to the invention, the hydrogenated phospholipid is at least partly present in an ortho-rhombic lamellar crystalline structure, wherein the wax is incorporated in the ortho-rhombic lamellar crystalline structure and/or is attached to the ortho-rhombic lamellar crystalline structure in the concentrate according to the invention.

For the concentrate according to the invention, all the advantages described above for the cosmetic product according to the invention are analogous or identical, which also applies to the embodiments and arrangements described above in the cosmetic product according to the invention. A particular advantage of the concentrate according to the invention lies in that fact that a plurality of differently composed cosmetic products can be produced from a single concentrate, and that the handling and the transport of the concentrate in comparison a plurality of cosmetic products filled in small units is particularly economically favorable and, moreover, is also simplified.

A further embodiment of the concentrate according to the invention proposes that the aqueous system has at least one cosmetic active ingredient. This embodiment of the concentrate according to the invention has the additional advantage, in addition to the advantages already described above, that for active ingredients which are sensitive to degradation, these active substances are fed to the aqueous system immediately before the preparation and are mixed with the concentrate, so that for cosmetic products of this type, which are provided with particularly sensitive cosmetic active ingredients, these cosmetic products are only produced immediately before shipment.

The present invention furthermore relates to the use of the above-described cosmetic product according to the invention and to the concentrate according to the invention which has also been set forth above.

In particular, the product according to the invention or the concentrate according to the invention is used for adjuvant care, for the prevention and/or treatment in infected, irritated or diseased skin, in particular in psoriasis, endogene eczema, radiation damage, light dermatosis, perlèche, actinic keratosis, contact dermatitis, seborrheic dermatitis, diaper dermatitis, couperosis, decubitus, ichthyosis, herpes labialis, lentigo, periooral dermatitis, scabies, urticaria, first-degree burns, and/or of such skin disorders, which are generated by ionizing radiation and/or by cytostatics.

Depending on the above-mentioned use, the active compounds are used for this purpose in the product according to the invention or in the concentrate according to the invention, said active compounds are mentioned above in patent claim 17 and are highlighted in the accompanying description as preferred active ingredients, so that reference is made to avoiding repetitions.

In order to detect the repeatedly described ortho-rhombic lamellar crystalline structure in the cosmetic product according to the invention and the concentrate according to the invention, an X-ray structure analysis is performed, as will be described in more detail below in the exemplary embodiments.

Depending on the respective ratio of hydrogenated phospholipid to wax, the phase transition temperature of the hydrogenated phospholipid present at least partly in an ortho-rhombic lamellar crystalline structure to which the wax is incorporated and/or attached varies between 72° C. and 95° C.

The term and/or used in this description is to be understood both as additive and as an alternative. Likewise, a formulation used in the singular also covers the plural, and a formulation used in the plural covers the singular.

Rice bran wax is also often referred to as rice wax.

Advantageous further developments of the cosmetic product according to the invention and of the concentrate according to the invention are specified in the subclaims.

### BRIEF DESCRIPTION OF THE DRAWINGS

The cosmetic product according to the invention is explained in more detail in conjunction with the schematic drawings according to FIGS. 1 to 3, 4, 5, 5A and 6 of the two efficacy studies, and the exemplary embodiments 1 to 12. They show:

FIG. 1 is a schematic illustration of an ortho-rhombic lamellar crystalline structure;

FIG. 2 is a schematic illustration of a single plane of the ortho-rhombic lamellar crystalline structure depicted in FIG. 1;

FIG. 3 is a schematic illustration of a single plane of a hexagonal lamellar crystalline structure;

FIGS. 4, 5 and 5A are the X-ray structure analysis results; and

FIG. 6 is the result of a first efficacy study.

### DETAILED DESCRIPTION OF THE INVENTION

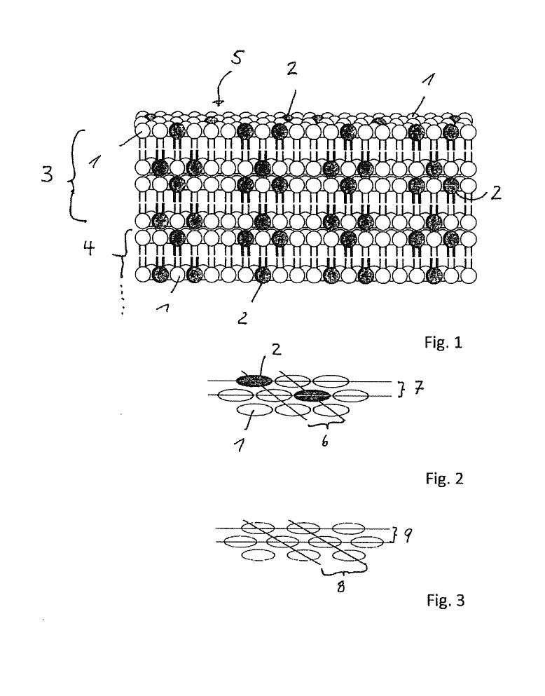

FIG. 1 schematically depicts an ortho-rhombic lamellar crystalline structure which forms the hydrogenated phospholipid and in particular the hydrogenated phosphatidylcholine under the manufacturing conditions as described in detail in the embodiments 1 to 12. The hydrogenated phospholipid molecules (exemplarily designated 1) are characterized by a white circle. On or in this structure are shown black wax molecules (exemplarily designated 2) attached and/or incorporated.

The structure shown in FIG. 1 has, for example, a first planar double membrane layer 3 and a planar second double membrane layer 4, which is shown only partially, wherein these two double membrane layers 3 and 4 are sandwiched one above the other.

The completely illustrated double membrane layer 3 consists of two layers, wherein the individual molecules of the hydrogenated phospholipid 1 and the wax 2 are aligned within the layers as shown in FIG. 1.

In FIG. 2, the ortho-rhombic lamellar crystalline structure is schematically depicted at a section in a single plane, which corresponds to the plane of the plane identified by 5 in FIG. 1 and thus represents a plan view of the lamellar structure shown in FIG. 1.

Viewed in the direction of this plane 5, for the ortho-rhombic lamellar crystalline structure shown in FIG. 2, the molecular distance designated by 6 is 3.71 Å, while the lateral distance of the molecules designated by 7 is 4.16 Å.

In contrast to FIG. 2, FIG. 3 shows a hexagonal lamellar crystalline structure. Here, the molecular distances 8 and 9 which correspond to the distances 6 and 7 of the structure shown in FIG. 2 with regard to their position are respectively 4.16 Å.

The above-noted differences in the molecular distances, on the one hand of 3.71 Å and 4.16 Å for the ortho-rhombic lamellar crystalline structure according to FIG. 2 and on the other hand of 4.16 Å (in both directions) on the hexagonal lamellar crystalline structure according to FIG. 3, can be clearly demonstrated by the X-ray structure analysis described in the following.

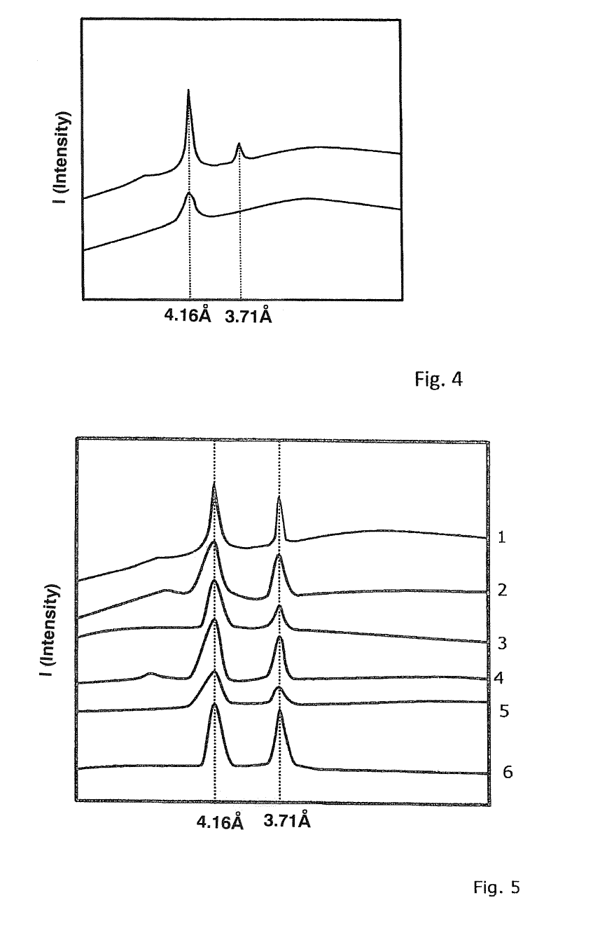

This proof is demonstrated by FIG. 4, which reflects the corresponding X-ray structure analysis result for the two formulations used in the first comparative efficacy study described in the following, designated there with formulation A (according to the invention) and comparison formulation B.

FIGS. 5 and 5A also show that all the compositions described in embodiments 1 to 12 form an ortho-rhombic lamellar crystalline structure, wherein the exemplary embodiments 1 to 6 and 7 to 12 are identified on the right-hand side of these two illustration by the reference numerals 1 to 6 and 7 to 12. All the X-ray structure analysis results shown there have two peaks, one at 3.71 Å and a second peak at 4.16 Å.

It should be noted for FIGS. 4, 5 and 5A that the intensity is not calibrated, since FIG. 4 is the reproduction of the two X-ray structure analysis results of the two formulations A and B compared with one another in the first efficacy study, and FIG. 5 is reproduction of the six X-ray structure analysis results for embodiments 1 to 6, each of which is summarized in a diagram. FIG. 5A graphically reproduces the results of the X-ray structure analysis of the exemplary embodiments 7 to 12.

Proof of the Ortho-Rhombic Structure

The ortho-rhombic lamellar crystalline structures listed in the present application and the hexagonal lamellar crystalline structure are detected as follows by X-ray structure analysis:

Apparatus: MARCCD-345X X-ray diffraction apparatus
rotating Cu anode X-ray generator, λ=1.5418 Å
Ray: Diameter: 1×1mm²
Transmission geometry: Beam perpendicular to the sample surface
Detection: Image area camera, 2300×2300 pixels, pixel size: 150×150 μm²
Sample detector distance: 250 mm
range of reticular distances: 50 Å to 2.7 Å, forward scattering vector S (=1/d): 0.020 to 0.37 Å⁻¹
Exposure time: 900 s.
T=21° C., 40% relative room humidity Data analysis:
Subtraction of the background signal (air) measured outside the sample
Software: FIT2D

The samples examined, with a single peak at 4.16 Å in the X-ray diagram (FIG. 4), is characteristic of hexagonal crystalline lamellar structures, while those samples in which the X-ray diagram shows a first peak at 4.16 Å and a second peak at 3.71 Å (FIGS. 4, 5 and 5A) are characteristic of ortho-rhombic lamellar crystalline structures. Accordingly, formulation A (according to the invention) has an ortho-rhombic lamellar crystalline structure and the comparative formulation B has a hexagonal crystalline structure.

First Comparative Efficacy Study

A first comparative efficacy study was performed between an embodiment of the cosmetic product of the invention having an ortho-rhombic lamellar crystalline structure and a formulation having a hexagonal lamellar structure (comparative formulation).

The hydrogenated phospholipid used in this comparison contains a concentration of hydrogenated phosphatidylcholine of 93% by weight, as in the case of the embodiment examples 1 to 12 described below, ±3% by weight, based on the weight of the hydrogenated phospholipid.

The composition of the formulation A according to the invention and the comparative formulation B was as follows:

| | | according | |
|---|---|---|---|
| | | to the invention | comparative |
| | | formulation A | formulation B in % |
| Phase | Ingredients | in % by weight | by weight |
| 1 | Hydrogenated | 1.50 | 1.50 |
| | phospholipid | | |
| 1 | Isostearyl isostearates | 15.00 | 15.00 |
| 1 | Rice bran wax | 1.20 | 0.10 |
| 1 | Glycerin 99.5% | 3.00 | 3.00 |
| 1 | Pentylene glycol | 5.00 | 5.00 |
| 2 | Water | 74.30 | 75.40 |
| | Σ | 100.00 | 100.00 |

The two formulations required for the first comparative efficacy study were manufactured according to the same manufacturing process.

Phase 1 was heated to 90° C. with uniform stirring. Phase 2 was then also heated to 90° C. At 90° C., phase 2 was added to phase 1 and then homogenized at 15,000 rpm using Ultra Turrax for 3 minutes. Subsequently, the mixture was cooled to 77° C. with homogenization (12,000 rpm by means of Ultra Turrax) at a cooling rate of 1.5° C./min.

The resulting predispersion was finely dispersed by means of a high-pressure homogenizer. Three cycles were required at 800 bar. The mixture was then cooled to 25° C. (cooling rate 1° C./min) with stirring.

The two formulations thus prepared were subjected to the in-process control described below. Both formulations were a homogeneous dispersion having evenly sized particles.

The comparative efficacy study was performed with 10 subjects. The subjects were women between 37 and 52 years of age. The average age was 45 years. There were no test-relevant diseases or skin changes in the subjects. The transepidermal water loss served as an efficacy criterion, which was measured and evaluated with the help of the Aquaflux AF 200 measuring device (manufacturer: Biox). The evaluation and documentation was performed using the 64 bit software system provided by BIOX.

On the first day of the study, four skin areas were identified in each subject at a distance of 5 cm from the elbow flexion of the right and left forearm on an area of 2×2 cm. On three of the four marked skin areas, deliberate barrier disorders were generated by irritation of the respective skin area with 0.0125 ml of a 15% sodium hydroxide solution. The skin showed no irritation or injury before the application of the sodium hydroxide solution. The sodium hydroxide solution was spread with a plastic spatula onto the respective skin area. After five minutes of exposure time, the sodium hydroxide solution was removed by rinsing with isotonic sodium chloride solution, after which the irritated skin area was dabbed and allowed to air dry for 20 minutes. This skin barrier disorder thus produced was then repeated identically once again for 20 minutes, but only for an exposure time of the sodium hydroxide solution of 3 minutes. The thus treated skin was rinsed, blotted and dried as described above.

After this irritation, the skin showed a slight swelling and redness in the sense of an erythema. Deeper skin damage, which go beyond the range of the epidermis, could not be ascertained. The transepidermal water loss was determined from all four skin areas (3 damaged and comparative area) and is shown as “Initial value” in the following table. The transepidermal water loss after injury had an average value of 45.07 g H₂O/m². The standard deviation was 4.2 g H₂O/m².

20 minutes after the second damage described above, the formulation A (0.01 ml, right forearm) according to the invention and the comparison formulation B (0.01 ml, left forearm) were applied to the irritated skin area by means of a microliter syringe on a damaged skin area on each arm and distributed with a spatula.

The other marked skin surfaces remained without treatment.

On the first day of the injury, 9 measurements of transepidermal water loss were performed at a time interval of 20 minutes.

On the morning of the second and the morning of the third day, the formulations to be compared were again applied to the corresponding skin areas in the manner described above.

On the second and third day after approximately five hours after further application of the formulations to be compared, transepidermal water losses were again measured. All results of the measurements are given in the table below.

TABLE 1

| | | | | |
|---|---|---|---|---|
| | | Average value of | | |
| | | transepidermal | | Average value of |
| | | H₂O loss | | transepidermal |
| | | formulation | Average value of | H₂O loss |
| | Transepidermal | according to the | transepidermal | comparison |
| | H₂O loss no | invention | H₂O loss without | formulation |
| Time | damage | Right forearm | treatment | Left forearm |

First day

| | | | | |
|---|---|---|---|---|
| Initial value | 8.94 | 43.61 | 48.95 | 46.53 |
| after | | | | |
| damage | | | | |
| 20 min after | 9.31 | 25.99 | 39.93 | 26.77 |
| application | | | | |
| 40 min after | 8.78 | 27.46 | 39.83 | 28.25 |
| application | | | | |
| 60 min after | 9.21 | 27.4 | 41.94 | 25.6 |
| application | | | | |
| 80 min after | 8.85 | 25.57 | 40.18 | 24.27 |
| application | | | | |
| 100 min | 8.91 | 23.34 | 41.9 | 24.75 |
| after | | | | |
| application | | | | |
| 120 min | 9.12 | 21.32 | 39.27 | 23.89 |
| after | | | | |
| application | | | | |
| 140 min | 9.18 | 19.41 | 38.89 | 25.27 |
| after | | | | |
| application | | | | |
| 160 min | 8.87 | 19.09 | 40.79 | 25.25 |
| after | | | | |
| application | | | | |

second day
(second application of the formulations to be compared)

| | | | | |
|---|---|---|---|---|
| 24 hours | 9.01 | 16.91 | 36.89 | 24.92 |
| after injury | | | | |

third day
(third application of the formulations to be compared)

| | | | | |
|---|---|---|---|---|
| 48 hours | 9.12 | 14.53 | 34.38 | 24.35 |
| after injury | | | | |

Table 1 above shows the measurement of transepidermal water loss in the first comparative efficacy study. On the first day, 20 minutes after the first application of the two formulations A and B to be compared, a clearly measurable decline in transepidermal water loss was observed. After 120 minutes after the first application on the first day, the transepidermal water loss in the formulation A according to the invention was lower than in the comparison formulation B, wherein the differences in the transepidermal water loss between the skin areas which were treated with the formulation A according to the invention and the skin areas which were treated with the conventional comparative formulation B, can be clearly seen from the measured values. This evidence intensified on the second day and on the third day so that formulation A according to the invention healed the barrier damage produced by the treatment with the sodium hydroxide solution substantially faster than the conventional comparison formulation B.

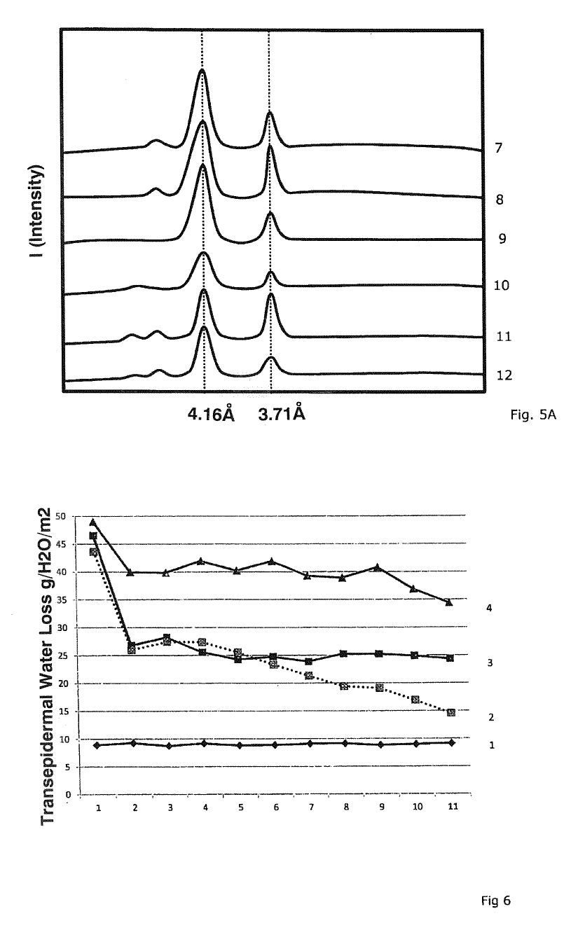

The results shown in Table 1 above are graphically shown in FIG. 6. Herein, the lower curve (denoted by 1) shows the transepidermal water loss of the non-damaged and untreated skin areas. The dotted curve (denoted by 2) represents the transepidermal water loss of the damaged skin areas subsequently treated with the formulation A according to the invention, while the illustrated curve represented by solid squares (denoted by 3) represents the transepidermal water loss of the damaged skin areas then treated with the conventional comparison formulation B. The uppermost curve (denoted by 4) represents the transepidermal water loss of the damaged but untreated skin areas.

An X-ray structure analysis was made of the formulations to be compared with regard to their composition quantified and with respect to their efficacy. The result of this X-ray structure analysis is shown in FIG. 4.

This clearly demonstrates that the formulation according to the invention has both a peak at 4.16 Å and at 3.71 Å (upper curve) and thus has ortho-rhombic lamellar crystalline structures while the conventional comparative formulation B has only a single peak at 4.16 Å (lower curve) and accordingly has a hexagonal lamellar crystalline structure.

Furthermore, the examples 1 to 12 described below were manufactured.

### EXAMPLES

In general, it should be noted with respect to the examples 1 to 12 that all the cooling processes described therein were performed with a cooling rate of 1.5° C./min ±0.5° C./min for all production processes.

In examples 1, 2, 4, 7, 8, 9 and 11, a solvent or active compound mixture of 4-t-butylcyclohexanol with pentylene glycol was used. This was the commercial product Symsitive, marketed by Symrise AG.

The in-process control described in all the examples was performed under 100-fold magnification using a light microscope (system microscope Olympus CH2, model CHT, Nikon Coolpix). The homogeneity of the respective dispersion and its uniformity were assessed visually.

The cosmetic products described in the following exemplary examples 1 to 12 were performed on a laboratory high-pressure homogenizer APV Gaulin Micron Lab 40.

### Example 1

Cream for the Sensitive, Allergy-Prone Skin

| Phase | Raw material | [%] |
|---|---|---|
| 1 | Hydrogenated phospholipid | 1.500 |
| 1 | Rice bran wax | 1.000 |
| 1 | Isostearly isostearates | 3.000 |
| 1 | Pentylene glycol | 1.250 |
| 1 | Glycerin | 0.750 |
| 2 | Water | 17.500 |
| 3 | Moringa butter | 2.500 |
| 3 | Acrylate/vinyl isodecanoate cross-polymer | 0.270 |
| 3 | Isostearly isostearates | 8.000 |
| 3 | Olus oil | 5.000 |
| 4 | Xanthan gum | 0.100 |
| 4 | Water | 54.030 |
| 4 | Pentylene glycol | 0.750 |
| 4 | Hydroxyethyl cellulose | 0.150 |
| 4 | L-serine | 0.700 |
| 4 | Sodium polyaspertates | 0.200 |
| 4 | L-glycine | 0.300 |
| 4 | 1,2 hexanediol | 1.500 |
| 5 | Sodium hydroxide solution 30% | 0.30 |
| 6 | 4-t-butylcyclohexanol, pentylene glycol (symsitive) | 1.200 |
| | Σ | 100.000 |

Phase 1 was heated to 90° C. with uniform stirring. Phase 2 was then also heated to 90° C. At 90° C., phase 2 was added to phase 1 and then homogenized at 15,000 rpm using Ultra Turrax for 3 minutes. Subsequently, the mixture was cooled to 77° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles were required at 800 bar. The mixture was then cooled to 35° C. with stirring. The pre-phase mixture was now forced high pressure homogenized at 300 bar (1 cycle) and temporarily stored in a separate vessel at 35° C. In the batch vessel, phase 3 was then heated with stirring to 50° C. and stirred until the lipid had completely melted. In a further container, the phase 4 was stirred at 50° C. while dispersing until a clear gel dispersion was obtained. Phase 4 was then added to phase 3 and then homogenized for 7 minutes at 20,000 rpm by means of Ultra Turrax at 50° C.

Subsequently, the phase 5 was added to the mixture of phase 3+4 and homogenized at 12,000 rpm by means of Ultra Turrax for 2 minutes. Subsequently, phase 6 was added to the mixture of phase 3+4+5 and homogenized down to the target temperature of 35° C. during a continuous homogenization process at 12,000 rpm by means of Ultra Turrax. This process took 12 minutes. Subsequently, the predispersion from phases 1+2 was added to the mixture from phases 3+4+5+6 and homogenized at 20,000 rpm with continuous stirring for 10 minutes. Finally, the mixture was cooled down to 25° C. under continuous homogenization at 12,000 rpm. This process took 19 minutes.

### Example 2

Body Cream Lotion for Over-Sensitive, Allergy-Prone Skin

| Phase | Raw material | [%] |
|---|---|---|
| 1 | Hydrogenated phospholipid | 1.200 |
| 1 | Rice bran wax | 1.200 |
| 1 | Isostearly isostearates | 1.600 |
| 1 | Pentylene glycol | 1.000 |
| 1 | Glycerin | 1.600 |
| 2 | Water | 13.400 |
| 3 | Moringa oil | 4.000 |
| 3 | Acrylate/vinyl isodecanoate cross-polymer | 0.150 |
| 3 | Isostearly isostearates | 8.000 |
| 3 | Caprylic/capric triglycerides | 15.000 |
| 4 | Xanthan gum | 0.100 |
| 4 | Water | 47.280 |
| 4 | Pentylene glycol | 1.250 |
| 4 | Hydroxyethyl cellulose | 0.150 |
| 4 | L-serine | 0.700 |
| 4 | Sodium polyaspertates | 0.200 |
| 4 | L-glycine | 0.300 |
| 4 | 1,2 hexanediol | 1.500 |
| 5 | Sodium hydroxide solution 30% | 0.17 |
| 6 | 4-t-butylcyclohexanol, pentylene glycol (Symsitive) | 1.200 |
| | Σ | 100.0 |

Phase 1 was heated to 90° C. with uniform stirring. Phase 2 was then also heated to 90° C. At 90° C., phase 2 was added to phase 1 and then homogenized at 15,000 rpm using Ultra Turrax for 6 minutes. Subsequently, the mixture was cooled to 77° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Five cycles were required at 800 bar. The mixture was then cooled to 35° C. with stirring. The pre-phase mixture was now forced high pressure homogenized at 500 bar (1 cycle) and temporarily stored in a separate vessel at 35° C. The phase 3 was then heated to 40° C. while stirring in the batch vessel. In a further container, the phase 4 was stirred at 40° C. while dispersing until a clear gel dispersion was obtained. Phase 4 was then added to phase 3 and then homogenized for 4 minutes at 12,000 rpm by means of Ultra Turrax at 40° C.

Subsequently, the phase 5 was added to the mixture of phase 3+4 and homogenized at 10,000 rpm by means of Ultra Turrax for 2 minutes. Subsequently, the phase 6 was added to the mixture of phase 3+4+5 and homogenized at 12,000 rpm by means of Ultra Turrax for 2 minutes. The mixture was then homogenized down to the target temperature of 35° C. during a continuous homogenization process at 12,000 rpm by means of Ultra Turrax. This process took 6 minutes. Subsequently, the predispersion from phases 1+2 was added to the mixture from phases 3+4+5+6 and homogenized at 15,000 rpm with continuous stirring for 5 minutes. Finally, the mixture was cooled down to 25° C. under continuous homogenization at 12,000 rpm. This process took 15 minutes.

### Example 3

Protective Cream when Exposed to Extreme Environmental Influences

| Phase | Raw material | [%] |
|---|---|---|
| 1 | Hydrogenated phospholipid | 1.500 |
| 1 | Rice bran wax | 1.000 |
| 1 | Isostearly isostearates | 3.000 |
| 1 | Pentylene glycol | 1.250 |
| 1 | Glycerin | 0.750 |
| 2 | Water | 17.500 |
| 3 | Moringa butter | 2.000 |
| 3 | Acrylate/vinyl isodecanoate cross-polymer | 0.350 |
| 3 | Isostearly isostearates | 4.000 |
| 3 | Tinosorb S (Bis-ethylhexyloxyphenol methoxyphenyl | 1.500 |
| | triazines) | |
| 3 | Uvinul A Plus (diethylamino hydroxybenzoyl hexyl | 1.500 |
| | benzoate) | |
| 3 | Ronacare AP (bis-ethylhexyl hydroxydimethoxy | 1.000 |
| | benzylmalonate) | |
| 4 | Water | 52.850 |
| 4 | Pentylene glycol | 0.750 |
| 4 | Hyaluronic acid 400 kDa | 0.400 |
| 4 | Glycerin | 8.000 |
| 4 | L-glycine | 0.500 |
| 4 | 1,2 hexanediol | 1.500 |
| 5 | Troxerutin | 0.250 |
| 6 | Sodium hydroxide solution 30% | 0.400 |
| | Σ | 100.000 |

Phase 1 was heated to 90° C. with uniform stirring. Phase 2 was then also heated to 90° C. At 90° C., phase 2 was added to phase 1 and then homogenized at 15,000 rpm using Ultra Turrax for 3 minutes. Subsequently, the mixture was cooled to 77° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles were required at 800 bar. The mixture was then cooled to 35° C. with stirring. The pre-phase mixture was now forced high pressure homogenized at 300 bar (1 cycle) and temporarily stored in a separate vessel at 35° C. The phase 3 is then heated in the batch vessel to 80° C. with stirring and stirring is continued until the UV filters are completely melted. In a further container, the phase 4 was stirred at 80° C. while dispersing until a clear gel dispersion was obtained. Phase 4 was then added to phase 3 and then homogenized for 8 minutes at 24,000 rpm by means of Ultra Turrax at 80° C.

Subsequently, the phase 5 was added to the mixture of phase 3+4 and homogenized at 20,000 rpm by means of Ultra Turrax for 4 minutes. Subsequently, the phase 6 was added to the mixture of phase 3+4+5 and homogenized at 20,000 rpm by means of Ultra Turrax for 5 minutes. The mixture was then homogenized down to the target temperature of 35° C. during a continuous homogenization process at 12,000 rpm by means of Ultra Turrax. This process took 30 minutes. Subsequently, the predispersion from phases 1+2 was added to the mixture from phases 3+4+5+6 and homogenized at 20,000 rpm with continuous stirring for 5 minutes. Finally, the mixture was cooled down to 25° C. under continuous homogenization at 18,000 rpm. This process took 17 minutes.

### Example 4

Care Cream for Accompanying Adjuvant Treatment for Neurodermatitis of Diseased Skin

| Phase | Raw material | [%] |
|---|---|---|
| 1 | Hydrogenated phospholipid | 1.800 |
| 1 | Rice bran wax | 1.800 |
| 1 | Isostearly isostearates | 2.400 |
| 1 | Pentylene glycol | 1.500 |
| 1 | Glycerin | 1.800 |
| 2 | Water | 20.700 |
| 3 | Moringa butter | 6.000 |
| 3 | Sodium carbomer | 0.180 |
| 3 | Boswellic acid 20% | 2.500 |
| 3 | Caprylic/capric triglycerides | 6.000 |
| 4 | Xanthan gum | 0.100 |
| 4 | Water | 45.170 |
| 4 | Pentylene glycol | 1.000 |
| 4 | Hydroxyethyl cellulose | 0.150 |
| 4 | L-serine | 0.700 |
| 4 | Niacinamide | 4.000 |
| 4 | Sodium polyaspertates | 0.200 |
| 4 | L-glycine | 0.300 |
| 4 | 1,2 hexanediol | 1.500 |
| 4 | Butylene glycol, pentylene glycol, hydroxyphenyl | 1.000 |
| | propamidobenzoic acid (Symcalmine) | |
| 5 | 4-t-butylcyclohexanol, pentylene glycol (symsitive) | 1.200 |
| | Σ | 100.000 |

Phase 1 was heated to 90° C. with uniform stirring. Phase 2 was then also heated to 90° C. At 90° C., phase 2 was added to phase 1 and then homogenized at 15,000 rpm using Ultra Turrax for 8 minutes. Subsequently, the mixture was cooled to 77° C. under homogenization (12,000 rpm by means of Ultra Turrax) and subjected to the in-process control described at the beginning.

The mixture thus prepared had an inhomogeneous dispersion with unequally large particles.

For this reason, the mixture was again heated to 90° C. with stirring and then homogenized at 18,000 rpm using Ultra Turrax for 5 minutes and the mixture was cooled to 77° C. with homogenization (18,000 rpm by means of Ultra Turrax).

The new in-process control gave a homogeneous dispersion with uniformly large particles.

The resulting predispersion was finely dispersed by means of a high-pressure homogenizer. Five cycles were required at 800 bar. The mixture was then cooled to 35° C. with stirring. The pre-phase mixture was now forced high pressure homogenized at 600 bar (1 cycle) and temporarily stored in a separate vessel at 35° C. The phase 3 was then heated to 50° C. while stirring in the batch vessel. In a further container, the phase 4 was stirred at 50° C. while dispersing until a clear gel dispersion was obtained. Phase 4 was then added to phase 3 and then homogenized for 3 minutes at 18,000 rpm by means of Ultra Turrax at 50° C.

Subsequently, the phase 5 was added to the mixture of phase 3+4 and homogenized at 20,000 rpm by means of Ultra Turrax for 5 minutes. The mixture was then homogenized down to the target temperature of 35° C. during a continuous homogenization process at 15,000 rpm by means of Ultra Turrax. This process took 14 minutes. Subsequently, the predispersion from phases 1+2 was added to the mixture from phases 3+4+5 and homogenized at 20,000 rpm with continuous stirring for 7 minutes. Finally, the mixture was cooled down to 25° C. under continuous homogenization at 20,000 rpm. This process took 18 minutes.

### Example 5

Sunscreen for Skin Tending to PLD (Polymorphic Light Dermatosis)

| Phase | Raw material | [%] |
|---|---|---|
| 1 | Hydrogenated phospholipid | 1.500 |
| 1 | Ilex Paraguariensis leaf wax | 1.000 |
| 1 | Caprylic/capric triglycerides | 2.000 |
| 1 | Pentylene glycol | 1.500 |
| 1 | Glycerin | 1.000 |
| 2 | Water | 20.700 |
| 3 | Octocrylene | 7.000 |
| 3 | PVP/Eicosene Copolymer | 0.100 |
| 3 | Acrylate/vinyl isodecanoate cross-polymer | 0.350 |
| 3 | Tinosorb S (Bis-ethylhexyloxyphenol | 3.800 |
| | methoxyphenyl triazines) | |
| 3 | Uvinul A Plus (diethylamino hydroxybenzoyl | 3.800 |
| | hexyl benzoate) | |
| 3 | Ronacare AP (bis-ethylhexyl hydroxydimethoxy | 1.000 |
| | benzylmalonate) | |
| 3 | C12-15 alkyl benzoates | 2.000 |
| 3 | Dicaprylyl carbonates | 4.000 |
| 4 | Xanthan gum | 0.100 |
| 4 | Water | 47.100 |
| 4 | Pentylene glycol | 1.000 |
| 4 | Hydroxyethyl cellulose | 0.150 |
| 4 | 1,2 hexanediol | 1.500 |
| 5 | Sodium hydroxide solution 30% | 0.400 |
| | Σ | 100.000 |

Phase 1 was heated to 86° C. with uniform stirring. Phase 2 was then also heated to 86° C. At 86° C., phase 2 was added to phase 1 and then homogenized at 15,000 rpm using Ultra Turrax for 4 minutes. Subsequently, the mixture was cooled to 75° C. under homogenization (10,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles were required at 800 bar. The mixture was then cooled to 35° C. with stirring. The pre-phase mixture was now forced high pressure homogenized at 300 bar (1 cycle) and temporarily stored in a separate vessel at 35° C. The phase 3 is then heated in the batch vessel to 80° C. with stirring and stirring continued until the UV filters are completely melted. In a further container, the phase 4 was stirred at 80° C. while dispersing until a clear gel dispersion was obtained. Phase 4 was then added to phase 3 and then homogenized for 10 minutes at 24,000 rpm by means of Ultra Turrax at 80° C.

Subsequently, the phase 5 was added to the mixture of phase 3+4 and homogenized at 18,000 rpm by means of Ultra Turrax for 5 minutes. The mixture was then homogenized down to the target temperature of 35° C. during a continuous homogenization process at 24,000 rpm by means of Ultra Turrax. This process took 34 minutes. Subsequently, the predispersion from phases 1+2 was added to the mixture from phases 3+4+5 and homogenized at 24,000 rpm with continuous stirring for 7 minutes. Finally, the mixture was cooled down to 25° C. under continuous homogenization at 15,000 rpm. This process took 16 minutes.

### Example 6

Lip Care Cream for Herpes Inclination

| Phase | Raw material | [%] |
|---|---|---|
| 1 | Hydrogenated phospholipid | 2.000 |
| 1 | Carnauba wax | 2.000 |
| 1 | Isostearly isostearates | 2.500 |
| 1 | Pentylene glycol | 1.800 |
| 1 | Glycerin | 3.000 |
| 2 | Water | 23.200 |
| 3 | Moringa butter | 2.000 |
| 3 | Acrylate/vinyl isodecanoate cross-polymer | 0.150 |
| 3 | Isostearly isostearates | 4.000 |
| 3 | Tinosorb S (Bis-ethylhexyloxyphenol methoxyphenyl | 2.800 |
| | triazines) | |
| 3 | Uvinul A Plus (diethylamino hydroxybenzoyl hexyl | 2.800 |
| | benzoate) | |
| 3 | Ronacare AP (bis-ethylhexyl hydroxydimethoxy | 1.000 |
| | benzylmalonate) | |
| 4 | Water | 42.530 |
| 4 | Xanthan gum | 0.100 |
| 4 | Pentylene glycol | 0.200 |
| 4 | Glycerin | 8.000 |
| 4 | 1,2 hexanediol | 1.500 |
| 5 | Troxerutin | 0.250 |
| 6 | Sodium hydroxide solution 30% | 0.17 |
| | Σ | 100.000 |

Phase 1 was heated to 92° C. with uniform stirring. Phase 2 was then also heated to 92° C. At 92° C., phase 2 was added to phase 1 and then homogenized at 15,000 rpm using Ultra Turrax for 12 minutes. Subsequently, the mixture was cooled to 77° C. under homogenization (13,000 rpm by means of Ultra Turrax) and subjected to the in-process control described at the beginning.

The mixture thus prepared had an inhomogeneous dispersion with unequally large particles.

For this reason, the mixture was again heated to 92° C. with stirring and then homogenized at 18,000 rpm using Ultra Turrax for 9 minutes and the mixture was cooled to 77° C. with homogenization (18,000 rpm by means of Ultra Turrax). The new in-process control gave a homogeneous dispersion with uniformly large particles.

The resulting predispersion was finely dispersed by means of a high-pressure homogenizer. Three cycles were required at 800 bar. The mixture was then cooled to 35° C. with stirring. The pre-phase mixture was now forced high pressure homogenized at 700 bar (1 cycle) and temporarily stored in a separate vessel at 35° C.

The phase 3 is then heated in the batch vessel to 80° C. with stirring and stirring continued until the UV filters are completely melted. In a further container, the phase 4 was stirred at 80° C. while dispersing until a clear gel dispersion was obtained. Phase 5 was then added to phase 4 with continuous stirring and stirring was continued until a clear dispersion had formed. Phase 4 was then added to phase 3 and then homogenized for 5 minutes at 18,000 rpm by means of Ultra Turrax at 80° C.

Subsequently, the phase 6 was added to the mixture of phase 3+4+5 and homogenized at 24,000 rpm by means of Ultra Turrax for 5 minutes. The mixture was then homogenized down to the target temperature of 35° C. during a continuous homogenization process at 24,000 rpm by means of Ultra Turrax. This process took 19 minutes. Subsequently, the predispersion from phases 1+2 was added to the mixture from phases 3+4+5+6 and homogenized at 24,000 rpm with continuous stirring for 11 minutes. Finally, the mixture was cooled down to 25° C. under continuous homogenization at 18,000 rpm. This process took 19 minutes.

Also, from these embodiments 1 to 6, X-ray structure analyses were performed under the conditions described above. The result of this X-ray structure analysis is summarized in FIG. 5.

This clearly proves that the formulations according to the invention according to examples 1 to 6 all have a peak at 4.16 Å and at 3.71 Å, and thus all formulations contain ortho-rhombic lamellar crystalline structures.

### Example 7

Cream for the Skin that Tends to Hyperreactivity

| Phase | Raw material | [%] |
|---|---|---|
| 1 | Hydrogenated phospholipid | 1.750 |
| 1 | Carnauba wax | 0.950 |
| 1 | Ficus Cerifera wax | 0.620 |
| 1 | Isostearly isostearates | 4.500 |
| 1 | Pentylene glycol | 1.250 |
| 1 | Glycerol 99.5% | 0.750 |
| 2 | Water | 16.180 |
| 3 | Moringa butter | 5.000 |
| 3 | Acrylate/vinyl isodecanoate cross- | 0.280 |
| | polymer | |
| 3 | Isostearly isostearates | 1.000 |
| 3 | Olus oil | 5.000 |
| 4 | Xanthan gum | 0.100 |
| 4 | Water | 52.220 |
| 4 | Pentylene glycol | 0.750 |
| 4 | Hydroxyethyl cellulose | 0.150 |
| 4 | Glycerol 99.5% | 6.000 |
| 4 | Sodium PCA Lsg. 50% | 0.200 |
| 4 | L-glycine | 0.300 |
| 4 | 1,2 hexanediol | 1.500 |
| 5 | Sodium hydroxide solution 30% | 0.300 |
| 6 | 4-t-butylcyclohexanol, Pentylene glycol | 1.200 |
| | (Symsitive) | |
| | Σ | 100.000 |

Phase 1 was heated to 85° C. with uniform stirring. Phase 2 was then also heated to 90° C. At 85° C., phase 2 was added to phase 1 and then homogenized at 15,000 rpm using Ultra Turrax for 5 minutes. Subsequently, the mixture was cooled to 78° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles are required at 800 bar. The mixture was then cooled to 35° C. with stirring and uniform homogenization at 9,000 rpm (Ultra Turrax). The pre-phase mixture was now forced high pressure homogenized at 350 bar (1 cycle) and temporarily stored in a separate vessel at 35° C. In the batch vessel, phase 3 was then heated with stirring to 55° C. and stirred until the lipid had completely melted. In a further container, the phase 4 was stirred at 55° C. while dispersing until a clear gel dispersion was obtained. Phase 4 was then added to phase 3 and then homogenized for 6 minutes at 20,000 rpm by means of Ultra Turrax at 50° C.

Subsequently, the phase 5 was added to the mixture of phase 3+4 and homogenized at 12,000 rpm by means of Ultra Turrax for 3 minutes. Subsequently, phase 6 was added to the mixture of phase 3+4+5 and homogenized down to the target temperature of 35° C. during a continuous homogenization process at 12,000 rpm by means of Ultra Turrax. This process took 16 minutes. Subsequently, the predispersion from phases 1+2 was added to the mixture from phases 3+4+5+6 and homogenized at 22,000 rpm with continuous stirring for 10 minutes. Finally, the mixture was cooled down to 25° C. under continuous homogenization at 12,000 rpm. This process took 21 minutes.

### Example 8

Cream for the Prevention of Hypertrophic Scarring

| Phase | Raw material | [%] |
|---|---|---|
| 1 | Hydrogenated phospholipid | 1.800 |
| 1 | Bamboo Oideae wax | 0.750 |
| 1 | Carnauba wax | 1.050 |
| 1 | Isostearly isostearates | 3.500 |
| 1 | Pentylene glycol | 1.500 |
| 1 | Glycerol 99.5% | 1.000 |
| 2 | Water | 20.400 |
| 3 | Moringa butter | 2.000 |
| 3 | Eusolex 9020 | 5.000 |
| 3 | Tinosorb S | 3.800 |
| 3 | Octocrylene | 7.000 |
| 3 | Uvinul A Plus | 3.800 |
| 3 | TiO2 | 1.500 |
| 3 | Acrylate/vinyl isodecanoate cross- | 0.250 |
| | polymer | |
| 3 | Finsolv TN | 6.000 |
| 3 | Olus oil | 3.000 |
| 4 | Xanthan gum | 0.100 |
| 4 | Water | 24.480 |
| 4 | Pentylene glycol | 0.750 |
| 4 | Hydroxyethyl cellulose | 0.150 |
| 4 | Glycerol 99.5% | 3.000 |
| 4 | Ectoin | 1.000 |
| 4 | Madecassoside | 0.200 |
| 4 | 1,2 hexanediol | 1.500 |
| 5 | Sodium hydroxide solution 30% | 0.270 |
| 6 | 4-t-butylcyclohexanol, Pentylene glycol | 1.200 |
| | (Symsitive) | |
| 6 | Polyhexane (water, snail secretion filtrate, | 5.000 |
| | propylene glycol, glycerine, | |
| | glycosaminoglycans, allantoin, hydrolyzed | |
| | collagen, pyridoxine hcl, poxethanol) | |
| | Σ | 100.000 |

Phase 1 was heated to 85° C. with uniform stirring. Phase 2 was then also heated to 90° C. At 85° C., phase 2 was added to phase 1 and then homogenized at 15,000 rpm using Ultra Turrax for 5 minutes. Subsequently, the mixture was cooled to 78° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles were required at 800 bar. The mixture was then cooled to 35° C. with stirring and uniform homogenization at 9,000 rpm (Ultra Turrax). The pre-phase mixture was now forced high pressure homogenized at 350 bar (1 cycle) and temporarily stored in a separate vessel at 35° C. The phase 3 was then heated in the batch vessel to 85° C. with stirring and stirring continued until the lipid UV filters were completely melted. In a further container, the phase 4 was stirred at 85° C. while dispersing until a clear gel dispersion was obtained. Phase 4 was then added to phase 3 and then homogenized for 6 minutes at 20,000 rpm by means of Ultra Turrax at 85° C.

Subsequently, the phase 5 was added to the mixture of phase 3+4 and homogenized at 18,000 rpm by means of Ultra Turrax for 3 minutes. The dispersion from phases 3+4+5 was then cooled to 50° C. at 18,000 rpm using Ultra Turrax. Subsequently, phase 6 was added to the mixture of phase 3+4+5 and homogenized down to the target temperature of 35° C. during a continuous homogenization process at 18,000 rpm by means of Ultra Turrax. This process took 20 minutes. Subsequently, the predispersion from phases 1+2 was added to the mixture from phases 3+4+5+6 and homogenized at 24,000 rpm with continuous stirring for 14 minutes. Finally, the mixture was cooled down to 25° C. under continuous homogenization at 18,000 rpm. This process took 17 minutes.

### Example 9

Cream for the Prevention of Actinic Keratoses

| Phase | Raw material | [%] |
|---|---|---|
| 1 | Hydrogenated phospholipid | 1.800 |
| 1 | Carnauba wax | 1.200 |
| 1 | Isostearly isostearates | 3.500 |
| 1 | Pentylene glycol | 1.500 |
| 1 | Glycerol 99.5% | 1.000 |
| 2 | Water | 21.000 |
| 3 | Eusolex 9020 | 5.000 |
| 3 | Tinosorb S | 3.800 |
| 3 | Octocrylene | 7.000 |
| 3 | Uvinul A Plus | 3.800 |
| 3 | TiO2 | 1.500 |
| 3 | Ronacare AP | 1.000 |
| 3 | Acrylate/vinyl isodecanoate cross- | 0.260 |
| | polymer | |
| 3 | Finsolv TN | 9.000 |
| 3 | Olus oil | 3.000 |
| 4 | Xanthan gum | 0.100 |
| 4 | Water | 28.310 |
| 4 | Pentylene glycol | 0.750 |
| 4 | Hydroxyethyl cellulose | 0.150 |
| 4 | Glycerol 99.5% | 3.000 |
| 4 | Ronacare Troxerutin | 0.250 |
| 4 | Spectrastate PHL | 1.600 |
| 5 | Sodium hydroxide solution 30% | 0.280 |
| 6 | 4-t-butylcyclohexanol, Pentylene glycol | 1.200 |
| | (Symsitive) | |
| | Σ | 100.000 |

Phase 1 was heated to 85° C. with uniform stirring. Phase 2 was then also heated to 90° C. At 85° C., phase 2 was added to phase 1 and then homogenized at 15,000 rpm using Ultra Turrax for 5 minutes. Subsequently, the mixture was cooled to 78° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles are required at 800 bar. The mixture was then cooled to 35° C. with stirring and uniform homogenization at 9,000 rpm (Ultra Turrax). The pre-phase mixture was now forced high pressure homogenized at 350 bar (1 cycle) and temporarily stored in a separate vessel at 35° C.

The phase 3 was then heated in the batch vessel to 85° C. with stirring and stirring continued until the lipid UV filters were completely melted. In a further container, the phase 4 was stirred at 85° C. while dispersing until a clear, yellowish gel dispersion was obtained. Phase 4 was then added to phase 3 and then homogenized for 5 minutes at 18,000 rpm by means of Ultra Turrax at 85° C.

Subsequently, the phase 5 was added to the mixture of phase 3+4 and homogenized at 18,000 rpm by means of Ultra Turrax for 5 minutes. The dispersion from phases 3+4+5 was then cooled to 50° C. at 18,000 rpm using Ultra Turrax. Subsequently, phase 6 was added to the mixture of phase 3+4+5 and homogenized down to the target temperature of 35° C. during a continuous homogenization process at 18,000 rpm by means of Ultra Turrax. This process took 17 minutes. Subsequently, the predispersion from phases 1+2 was added to the mixture from phases 3+4+5+6 and homogenized at 20,000 rpm with continuous stirring for 16 minutes. Finally, the mixture was cooled down to 25° C. under continuous homogenization at 18,000 rpm. This process took 14 minutes.

### Example 10

Care Cream for the Prevention of Light-Induced Skin Aging Symptoms

| Phase | Raw material | [%] |
|---|---|---|
| 1 | Hydrogenated phospholipid | 1.200 |
| 1 | Carnauba wax | 0.800 |
| 1 | Isostearly isostearates | 2.400 |
| 1 | Pentylene glycol | 1.000 |
| 1 | Glycerol 99.5% | 0.600 |
| 2 | Water | 14.000 |
| 3 | Moringa butter | 1.000 |
| 3 | Finsolv TN | 2.500 |
| 3 | Crodamol ISIS-LQ-(MV) | 1.000 |
| 3 | Myritol 312 | 3.000 |
| 3 | Tinosorb S | 1.500 |
| 3 | Uvinul A Plus | 1.500 |
| 3 | Stabylene 30 | 0.250 |
| 3 | Smartgel P110 | 0.300 |
| 3 | Amaze XT | 0.100 |
| 4 | osmosis water | 49.486 |
| 4 | RonaCare (R) Troxerutin | 0.250 |
| 4 | Glycerol 99% Ph. Eur. anhydrous | 8.000 |
| 4 | Raya Hyaluron LMW (50 kDa) | 0.100 |
| 4 | Hydrolite-5 | 1.000 |
| 5 | Osmosis water | 5.000 |
| 5 | Niacinamide PC | 2.000 |
| 6 | Hydrolite-6 | 0.300 |
| 7 | Spectrastat (TM) PHL | 1.600 |
| 8 | Ronacare AP | 1.000 |
| 9 | Sodium hydroxide 32% solution | 0.114 |
| | | 100.000 |

Phase 1 was heated to 85° C. with uniform stirring. Phase 2 was then also heated to 90° C. At 85° C., phase 2 was added to phase 1 and then homogenized at 15,000 rpm using Ultra Turrax for 5 minutes. Subsequently, the mixture was cooled to 78° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles were required at 800 bar. The mixture was then cooled to 35° C. with stirring and uniform homogenization at 9,000 rpm (Ultra Turrax). The pre-phase mixture was now forced high pressure homogenized at 350 bar (1 cycle) and temporarily stored in a separate vessel at 35° C.

The phase 3 was then heated in the batch vessel to 85° C. with stirring and stirring continued until the lipid UV filters were completely melted. In a further container, the phase 4 was stirred at 85° C. while dispersing until a clear, yellowish gel dispersion was obtained. Phase 4 was then added to phase 3 and then homogenized for 5 minutes at 18,000 rpm by means of Ultra Turrax at 85° C.

Subsequently, the phase 5 was added to the mixture of phase 3+4 and homogenized at 18,000 rpm by means of Ultra Turrax for 5 minutes. The dispersion from phases 3+4+5 was then cooled to 50° C. at 18,000 rpm using Ultra Turrax. Subsequently, phase 6 was added to the mixture of phase 3+4+5 and homogenized down to the target temperature of 35° C. during a continuous homogenization process at 18,000 rpm by means of Ultra Turrax. This process took 17 minutes. Subsequently, the predispersion from phases 1+2 was added to the mixture from phases 3+4+5+6 and homogenized at 20,000 rpm with continuous stirring for 16 minutes. Finally, the mixture is cooled down to 25° C. under continuous homogenization at 18,000 rpm. This process took 14 minutes.

### Example 11

Light Protection Cream for the Skin Tending to Polymorphous Light Dermatosis

| Phase | Raw material | [%] |
|---|---|---|
| 1 | Hydrogenated phospholipid | 1.500 |
| 1 | Carnauba wax | 1.500 |
| 1 | Isostearly isostearates | 2.900 |
| 1 | Pentylene glycol | 1.250 |
| 1 | Glycerol 99.5% | 0.600 |
| 2 | Water | 17.200 |
| 3 | Eusolex 9020 | 3.500 |
| 3 | Tinosorb S | 2.500 |
| 3 | Octocrylene | 10.000 |
| 3 | Uvinul A Plus | 2.500 |
| 3 | TiO2 | 2.500 |
| 3 | Acrylate/vinyl isodecanoate cross-polymer | 0.260 |
| 3 | Finsolv TN | 5.000 |
| 3 | Olus oil | 5.000 |
| 4 | Xanthan gum | 0.100 |
| 4 | Water | 34.260 |
| 4 | Hydroxyethyl cellulose | 0.150 |
| 4 | Glycerol 99.5% | 5.000 |
| 4 | Ronacare Troxerutin | 0.250 |
| 4 | Spectrastate PHL | 1.600 |
| 5 | Sodium hydroxide solution 30% | 0.280 |
| 6 | 4-t-butylcyclohexanol, Pentylene glycol (Symsitive) | 1.200 |
| 6 | Tetrahydrocurum cuminoid (THC) | 0.200 |
| 6 | Pentylene glycol | 0.750 |
| | Σ | 100.000 |

Phase 1 was heated to 85° C. with uniform stirring. Phase 2 was then also heated to 90° C. At 85° C., phase 2 was added to phase 1 and then homogenized at 15,000 rpm using Ultra Turrax for 5 minutes. Subsequently, the mixture was cooled to 78° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles were required at 800 bar. The mixture was then cooled to 35° C. with stirring and uniform homogenization at 9,000 rpm (Ultra Turrax). The pre-phase mixture was now forced high pressure homogenized at 350 bar (1 cycle) and temporarily stored in a separate vessel at 35° C.

The phase 3 was then heated in the batch vessel to 85° C. with stirring and stirring continued until the lipid UV filters were completely melted. In a further container, the phase 4 was stirred at 85° C. while dispersing until a clear, yellowish gel dispersion was obtained. Phase 4 was then added to phase 3 and then homogenized for 5 minutes at 16,000 rpm by means of Ultra Turrax at 85° C.

Subsequently, the phase 5 was added to the mixture of phase 3+4 and homogenized at 18,000 rpm by means of Ultra Turrax for 5 minutes. The dispersion from phases 3+4+5 was then cooled to 45° C. at 16,000 rpm using Ultra Turrax. Subsequently, the phase 6 was stirred at 45° C. to give a complete solution. Subsequently, this phase was added to the mixture of phase 3+4+5 and homogenized down to the target temperature of 35° C. during a continuous homogenization process at 16,000 rpm by means of Ultra Turrax. This process took 17 minutes. Subsequently, the predispersion from phases 1+2 was added to the mixture from phases 3+4+5+6 and homogenized at 16,000 rpm with continuous stirring for 16 minutes. Finally, the mixture was cooled down to 25° C. under continuous homogenization at 16,000 rpm. This process took 12 minutes.

### Example 12

Care Cream for the Over-Sensitive Skin, Tending to Dryness and Irritation

| Phase | Raw material | [%] |
|---|---|---|
| 1 | Hydrogenated phospholipid | 1.500 |
| 1 | Carnauba wax | 1.000 |
| 1 | Isostearly isostearates | 3.000 |
| 1 | Pentylene glycol | 1.250 |
| 1 | Glycerol 99.5% | 0.750 |
| 2 | Water | 17.500 |
| 3 | Crodamol ISIS-LQ-(MV) | 2.000 |
| 3 | Moringa butter | 1.500 |
| 3 | Cetiol SB 45 | 4.500 |
| 4 | Cegesoft PS6 | 7.500 |
| 4 | Soothex 20 CQ U/A | 2.500 |
| 5 | Antaron V-220 F | 0.100 |
| 5 | Stabylene 30 | 0.270 |
| 5 | Amaze XT | 0.050 |
| 6 | Osmosis water | 39.870 |
| 6 | Glycerol 99% Ph. Eur. anhydrous | 8.000 |
| 6 | Niacinamide PC | 2.000 |
| 6 | Natrosol 250GR | 0.150 |
| 6 | Symcalmin | 1.000 |
| 7 | Symsitive 1609 | 0.400 |
| 8 | Osmosis water | 2.000 |
| 8 | L-serine | 0.700 |
| 8 | Glycine (L) | 0.300 |
| 8 | Sodium polyaspartate C-LC/SD-PC | 0.100 |
| 9 | Spectrastat (TM) PHL | 1.600 |
| 9 | Hydrolite-6 | 0.300 |
| 10 | Sodium hydroxide 32% solution | 0.160 |
| | | 100.000 |

Phase 1 was heated to 85° C. with uniform stirring. Phase 2 was then also heated to 90° C. At 85° C., phase 2 was added to phase 1 and then homogenized at 15,000 rpm using Ultra Turrax for 5 minutes. Subsequently, the mixture was cooled to 78° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles were required at 800 bar. The mixture was then cooled to 35° C. with stirring and uniform homogenization at 9,000 rpm (Ultra Turrax). The pre-phase mixture was now forced high pressure homogenized at 350 bar (1 cycle) and temporarily stored in a separate vessel at 35° C.

The phase 3 was then heated in the batch vessel to 50° C. with stirring and stirring continued until the lipid components were completely melted. In a further container, the phase 6 was also brought to 50° C. with stirring and stirred until a clear gel dispersion was obtained.

Phases 4 and 5 were then added to phase 3 with stirring and directly thereafter phase 6 was added to the phase mixture. The mixture was then homogenized at 50° C. for 18 minutes at 18,000 rpm using Ultra Turrax for 5 minutes.

Subsequently, the phases 7, 8 and 9 were added to the mixture of phase 3+4+5+6 and homogenized at 18,000 rpm by means of Ultra Turrax for 8 minutes. The dispersion from phases 3+4+5+6+7+8+9 was then cooled to 45° C. at 18,000 rpm using Ultra Turrax. Subsequently, phase 10 was added to the mixture of phase 3+4+5+6+7+8+9 and homogenized down to the target temperature of 35° C. during a continuous homogenization process at 18,000 rpm by means of Ultra Turrax. This process took 14 minutes. Subsequently, the predispersion from phases 1+2 was added to the mixture from phases 3+4+5+6+7+8+9+10 and homogenized at 18,000 rpm with continuous stirring for 18 minutes. Finally, the mixture was cooled down to 25° C. under continuous homogenization at 18,000 rpm. This process took 11 minutes.

Second Efficacy Study

A second efficacy study was performed as a capsaicin sensitivity short study on five subjects, wherein a conventional product C formulated as a standard emulsion, a product D formulated according to the invention, in which the hydrogenated phospholipid was at least partially present in an ortho-rhombic lamellar crystalline structure, and a further conventional product E with hexagonal lamellar crystalline structure of the phospholipid were compared with one another. All three products C to E had an identical concentration of 4-t-butylcyclohexanol.

For this short study, five subjects (4 male, 1 female) with an average age of 39 years were used.

First, the left and right nasolabial folds of each subject were cleaned with a 2% sodium lauryl sulfate solution. For this purpose, 0.2 ml of the 2% sodium lauryl sulfate solution was applied to the left and right nasolabial folds and was massaged gently for 5 seconds per test field. Subsequently, the surfaces were carefully washed for two minutes under running warm water (temperature 35° C.±2° C.). Care was taken that the wash solution was completely removed. Subsequently, the correspondingly characterized nasolabial fold regions were dried by means of a soft, commercially available paper towel by careful, uniform dabbing. After a further 10 minutes, 0.05 g each of the products C and D (left and right of the nose) were applied and gently massaged.

After 8 to 10 minutes, 0.02 g of a liquid capsaicin extract was applied by pipette to all test fields, wherein this capsaicin liquid extract to be purchased was diluted 3% in a ratio of 1:10 with sunflower oil.

After 3 minutes, each subject assessed subjectively, but without influence by fellow test subjects, the intensity of burning and stinging, with the meaning zero=no burning/stinging, 1=mild burning/stinging, 2=moderate burning/stinging and 3=painful or very unpleasant burning/stinging.

After four days, the short study was repeated with the same five subjects, wherein the only difference to the previously described first part of the short study was not the products C and D compared with each other, but rather the products D and E.

The product C had the following ingredients and was prepared as follows:

Ingredients of the Formulation C

| Phase | Raw material | [%] |
|---|---|---|
| 1 | Emulgin SML 20 | 1.800 |
| 1 | Caprylic/capric triglycerides | 18.000 |
| 1 | Pentylene glycol | 5.000 |
| 1 | Glycerol 99.5% | 1.000 |
| 1 | Acrylate/vinyl isodecanoate cross-polymer | 0.300 |
| 1 | Sodium carbomer | 0.300 |
| 2 | Water | 72.400 |
| 3 | 4-t-butylcyclohexanol, Pentylene glycol (Symsitive) | 1.200 |
| | Σ | 100.000 |

Phase 1 was heated to 25° C. with uniform stirring. The gel bodies were dispersed uniformly in the phase. Phase 2 was then also heated to 25° C. Phase 2 was then added to phase 1 and then homogenized at 16,000 rpm using Ultra Turrax for 5 minutes and subjected to microscopic in-process control.

The resulting mixture had a homogeneous dispersion with uniformly smallest particles.

Subsequently, the phase 3 was added to the mixture of phase 1+2 and homogenized at 19,000 rpm by means of Ultra Turrax for 5 minutes. After this time, an even dispersion had developed.

The product D had the following ingredients and was prepared as follows:

| Phase | Raw material | [%] |
|---|---|---|
| 1 | Hydrogenated phospholipid | 1.800 |
| 1 | Carnauba wax | 1.500 |
| 1 | Isostearly isostearates | 3.500 |
| 1 | Pentylene glycol | 1.500 |
| 1 | Glycerol 99.5% | 1.000 |
| 2 | Water | 20.700 |
| 3 | Moringa butter | 5.000 |
| 3 | Acrylate/vinyl isodecanoate cross-polymer | 0.280 |
| 3 | Isostearly isostearates | 8.000 |
| 4 | Xanthan gum | 0.100 |
| 4 | Water | 46.720 |
| 4 | Pentylene glycol | 0.750 |
| 4 | Hydroxyethyl cellulose | 0.150 |
| 4 | Glycerol 99.5% | 6.000 |
| 4 | 1,2 hexanediol | 1.500 |
| 5 | Sodium hydroxide solution 30% | 0.300 |
| 6 | 4-t-butylcyclohexanol, Pentylene glycol (Symsitive) | 1.200 |
| | Σ | 100.000 |

Phase 1 was heated to 85° C. with uniform stirring. Phase 2 was then also heated to 90° C. At 85° C., phase 2 was added to phase 1 and then homogenized at 16,000 rpm using Ultra Turrax for 5 minutes. The mixture was then cooled to 78° C. under homogenization (14,000 rpm by means of Ultra Turrax) and subjected to microscopic in-process control.

The mixture thus prepared had a homogeneous dispersion with uniformly large particles.

The resulting predispersion was finely dispersed by means of a high-pressure homogenizer. Three cycles were required at 800 bar. The mixture was then cooled to 35° C. with stirring and uniform homogenization at 10,000 rpm (Ultra Turrax). The pre-phase mixture was now forced high pressure homogenized at 350 bar (1 cycle) and temporarily stored in a separate vessel at 35° C.

The phase 3 is then heated in the batch vessel to 50° C. with stirring and stirring continued until the lipid components are completely melted. In a further container, the phase 4 was stirred at 50° C. while dispersing until a clear, yellowish gel dispersion was obtained. Phase 4 was then added to phase 3 and then homogenized for 5 minutes at 19,000 rpm by means of Ultra Turrax at 85° C.

Subsequently, the phase 5 was added to the mixture of phase 3+4 and homogenized at 19,000 rpm by means of Ultra Turrax for 5 minutes. The dispersion from phases 3+4+5 was then cooled to 45° C. at 18,000 rpm using Ultra Turrax. Subsequently, phase 6 was added to the mixture of phase 3+4+5 and homogenized down to the target temperature of 35° C. during a continuous homogenization process at 18,000 rpm by means of Ultra Turrax. This process took 16 minutes. Subsequently, the predispersion from phases 1+2 was added to the mixture from phases 3+4+5+6 and homogenized at 20,000 rpm with continuous stirring for 18 minutes. Finally, the mixture was cooled down to 25° C. under continuous homogenization at 18,000 rpm. This process took 16 minutes.

The product E had the following ingredients and was prepared as follows:

| Phase | Raw material | [%] |
|---|---|---|
| 1 | Hydrogenated phospholipid | 1.800 |
| 1 | Caprylic/capric triglycerides | 5.000 |
| 1 | Pentylene glycol | 1.500 |
| 1 | Glycerol 99.5% | 1.000 |
| 2 | Water | 20.700 |
| 3 | Moringa butter | 5.000 |
| 3 | Acrylate/vinyl isodecanoate cross-polymer | 0.280 |
| 3 | Isostearly isostearates | 8.000 |
| 4 | Xanthan gum | 0.100 |
| 4 | Water | 46.720 |
| 4 | Pentylene glycol | 0.750 |
| 4 | Hydroxyethyl cellulose | 0.150 |
| 4 | Glycerol 99.5% | 6.000 |
| 4 | 1,2 hexanediol | 1.500 |
| 5 | Sodium hydroxide solution 30% | 0.300 |
| 6 | 4-t-butylcyclohexanol, Pentylene glycol (Symsitive) | 1.200 |
| | Σ | 100.000 |

Phase 1 was heated to 85° C. with uniform stirring. Phase 2 was then also heated to 90° C. At 85° C., phase 2 was added to phase 1 and then homogenized at 12,000 rpm using Ultra Turrax for 5 minutes. Subsequently, the mixture was cooled to 78° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Two cycles were required at 790 bar. The mixture was then cooled to 35° C. with stirring and uniform homogenization at 8,000 rpm (Ultra Turrax). The pre-phase mixture was now forced high pressure homogenized at 100 bar (1 cycle) and temporarily stored in a separate vessel at 35° C.

The phase 3 is then heated in the batch vessel to 50° C. with stirring and stirring continued until the lipid components are completely melted. In a further container, the phase 4 was stirred at 50° C. while dispersing until a clear, yellowish gel dispersion was obtained. Phase 4 was then added to phase 3 and then homogenized for 5 minutes at 19,000 rpm by means of Ultra Turrax at 85° C.

Subsequently, the phase 5 was added to the mixture of phase 3+4 and homogenized at 19,000 rpm by means of Ultra Turrax for 5 minutes. The dispersion from phases 3+4+5 was then cooled to 45° C. at 18,000 rpm using Ultra Turrax. Subsequently, phase 6 was added to the mixture of phase 3+4+5 and homogenized down to the target temperature of 35° C. during a continuous homogenization process at 18,000 rpm by means of Ultra Turrax. This process took 16 minutes. Subsequently, the predispersion from phases 1+2 was added to the mixture from phases 3+4+5+6 and homogenized at 16,000 rpm with continuous stirring for 14 minutes. Finally, the mixture is cooled down to 25° C. under continuous homogenization at 14,000 rpm. This process took 12 minutes.

The result of this second efficacy study is summarized in Tables 2 and 3 below.

TABLE 2

| | |
|---|---|
| | Product D |
| Product C | (ortho rhombic) |

| Test person | Burn | Sting | Burn | Sting |
|---|---|---|---|---|
| 1 | 2 | 3 | 1 | 1 |
| 2 | 2 | 3 | 1 | 1 |
| 3 | 3 | 3 | 2 | 2 |
| 4 | 2 | 3 | 1 | 1 |
| 5 | 3 | 3 | 2 | 1 |
| MW | 2.4 | 2.8 | 1.4 | 1.2 |

TABLE 3

| | |
|---|---|
| Product D | Product E |
| (ortho rhombic) | (hexagonal) |

| Test person | Burn | Sting | Burn | Sting |
|---|---|---|---|---|
| 1 | 1 | 1 | 2 | 1 |
| 2 | 1 | 2 | 2 | 2 |
| 3 | 2 | 2 | 2 | 2 |
| 4 | 1 | 1 | 2 | 1 |
| 5 | 1 | 1 | 2 | 2 |
| MW | 1.2 | 1.4 | 2.0 | 1.6 |

The above-described X-ray structure studies were conducted for the products D and E, with the result that the product D had two peaks at 4.16 Å and at 3.71 Å, thus containing an ortho-rhombic lamellar crystalline structure while the product E in the X-ray diagram showed only a single peak at 4.16 Å, and thus containing only a hexagonal lamellar crystalline structure.

The clear superiority of the formulation according to the invention is also clearly demonstrated by this second efficacy study.

Further studies on the stratum corneum, isolated from human skin

In order to investigate the physiological influence of compositions which on the one hand have the known hexagonal lamellar crystalline structures and on the other hand the ortho-rhombic lamellar crystalline structures frequently described in the present text, on the damaged skin in view of its influence on structural changes, the study described in the following was performed. For this purpose, reference is made in the literature, particularly in the publication by J. C. Garson et al in J. Invest. Dermatol. 96: 43-49, 1991, which recommends the use of delipidated stratum corneum, since this is closest to the in vivo skin condition.

The study was performed with an X-ray scattering synchrotron microbeam source that allows detection of small and wide angle scattering patterns on a stratum corneum sample with a spatial resolution of 1 micrometer. Stratum corneum was isolated from skin tissue, obtained by abdominal plastic surgery, by separation from the epidermis by immersion in water at 56° C. and subsequent tryptic digestion at 40° C. Subsequently, a portion was degreased of the intercellular stratum corneum lipids by 6 h extraction in chloroform/methanol (2:1).

Two products F and G described in more detail below were applied in an amount of 3 mg/cm² to the external side of the stratum corneum treated in the manner described above, and 0.5×3 mm large pieces were clamped into the holding device.

The technical parameters for this X-ray structure study, taking into account the previously mentioned publication, were as follows:
Experiments were performed on ESRF Beamline ID13
Energy: 13.6 keV, i.e. λ=0.9117 Å, mode 16 bunches
Beam size: cross-section 1.5(v)×2(h)μm²
Transmission geometry: Ray parallel to the surface of the stratum corneum
Detection: Frelon camera, pixel size: 50×50 μm²
Distance pattern detector: 133.9 mm
Range of reticular distances: 15 to 0.3 nm, e.g.: for the scatter vector S(=1/d): 0.070 to 3.3 nm⁻¹
Exposure time: 0.5 seconds
For each sample, the scattering data were collected along 3D scans, over the total thickness of the stratum corneum (3×40 positions at a distance of 2 μm), 3 scans/sample at 3 different positions were performed, T=22.5° C., relative humidity 30%.

Data analysis
right-angled integration along the narrow-angle equator (parallel to the surface of the stratum corneum)
   angular integration (−20° to +20°) along the wide-angle meridian (perpendicular to the surface of the stratum corneum)
   Data analysis with FIT2D software

The products F and G used in this study had the following ingredients:

| | | Formulation | |
|---|---|---|---|
| | | according to the | |
| | | invention | conventional |
| | | Product F | product G |
| Phase | Raw material | [%] | [%] |
| 1 | Hydrogenated phospholipid | 1.800 | 1.800 |
| 1 | Carnauba wax | 1.500 | 0.000 |
| 1 | Caprylic/capric triglycerides | 0.000 | 1.500 |
| 1 | Isostearly isostearates | 3.500 | 3.500 |
| 1 | Pentylene glycol | 1.500 | 1.500 |
| 1 | Glycerol 99.5% | 1.000 | 1.000 |
| 2 | Water | 20.700 | 20.700 |
| 3 | Moringa butter | 5.000 | 5.000 |
| 3 | Acrylate/vinyl isodecanoate | 0.280 | 0.280 |
| | cross-polymer | | |
| 3 | Isostearly isostearates | 8.000 | 8.000 |
| 4 | Xanthan gum | 0.100 | 0.100 |
| 4 | Water | 46.720 | 46.720 |
| 4 | Pentylene glycol | 0.750 | 0.750 |
| 4 | Hydroxyethyl cellulose | 0.150 | 0.150 |
| 4 | Glycerol 99.5% | 6.000 | 6.000 |
| 4 | 1,2 hexanediol | 1.500 | 1.500 |
| 5 | Sodium hydroxide solution 30% | 0.300 | 0.300 |
| 6 | 4-t-butylcyclohexanol, Pentylene | 1.200 | 1.200 |
| | glycol (Symsitive) | | |
| | Σ | 100.000 | 100.000 |

The product F has ortho-rhombic lamellar crystalline structures while the conventional product G has hexagonal lamellar crystalline structures.

The product F was prepared as follows:

Phase 1 was heated to 85° C. with uniform stirring. Phase 2 was then also heated to 90° C. At 85° C., phase 2 was added to phase 1 and then homogenized at 16,000 rpm using Ultra Turrax for 5 minutes. Subsequently, the mixture was cooled to 78° C. under homogenization (14,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Three cycles were required at 800 bar. The mixture was then cooled to 35° C. with stirring and uniform homogenization at 10,000 rpm (Ultra Turrax). The pre-phase mixture was now forced high pressure homogenized at 350 bar (1 cycle) and temporarily stored in a separate vessel at 35° C.

The phase 3 is then heated in the batch vessel to 50° C. with stirring and stirring continued until the lipid components were completely melted. In a further container, the phase 4 was stirred at 50° C. while dispersing until a clear, yellowish gel dispersion was obtained. Phase 4 was then added to phase 3 and then homogenized for 5 minutes at 19,000 rpm by means of Ultra Turrax at 85° C.

Subsequently, the phase 5 was added to the mixture of phase 3+4 and homogenized at 19,000 rpm by means of Ultra Turrax for 5 minutes. The dispersion from phases 3+4+5 was then cooled to 45° C. at 18,000 rpm using Ultra Turrax. Subsequently, phase 6 was added to the mixture of phase 3+4+5 and homogenized down to the target temperature of 35° C. during a continuous homogenization process at 18,000 rpm by means of Ultra Turrax. This process took 16 minutes. Subsequently, the predispersion from phases 1+2 was added to the mixture from phases 3+4+5+6 and homogenized at 20,000 rpm with continuous stirring for 18 minutes. Finally, the mixture was cooled down to 25° C. under continuous homogenization at 18,000 rpm. This process took 16 minutes.

The conventional product G was prepared as follows:

Phase 1 was heated to 85° C. with uniform stirring. Phase 2 was then also heated to 90° C. At 85° C., phase 2 was added to phase 1 and then homogenized at 12,000 rpm using Ultra Turrax for 5 minutes. Subsequently, the mixture was cooled to 78° C. under homogenization (12,000 rpm by means of Ultra Turrax) and subjected to the in-process control described at the beginning.

The mixture thus prepared had a homogeneous dispersion with uniformly large particles.

The resulting predispersion was finely dispersed by means of a high-pressure homogenizer. Two cycles were required at 790 bar. The mixture was then cooled to 35° C. with stirring and uniform homogenization at 8,000 rpm (Ultra Turrax). The pre-phase mixture was now forced high pressure homogenized at 100 bar (1 cycle) and temporarily stored in a separate vessel at 35° C.

The phase 3 is then heated in the batch vessel to 50° C. with stirring and stirring continued until the lipid components were completely melted. In a further container, the phase 4 was stirred at 50° C. while dispersing until a clear, yellowish gel dispersion was obtained. Phase 4 was then added to phase 3 and then homogenized for 5 minutes at 19,000 rpm by means of Ultra Turrax at 85° C.

Subsequently, the phase 5 was added to the mixture of phase 3+4 and homogenized at 19,000 rpm by means of Ultra Turrax for 5 minutes. The dispersion from phases 3+4+5 was then cooled to 45° C. at 18,000 rpm using Ultra Turrax. Subsequently, phase 6 was added to the mixture of phase 3+4+5 and homogenized down to the target temperature of 35° C. during a continuous homogenization process at 18,000 rpm by means of Ultra Turrax. This process took 16 minutes. Subsequently, the predispersion from phases 1+2 was added to the mixture from phases 3+4+5+6 and homogenized at 16,000 rpm with continuous stirring for 14 minutes. Finally, the mixture was cooled down to 25° C. under continuous homogenization at 14,000 rpm. This process took 12 minutes.

As a result of this investigation, it should be noted that the structures of the two applied products F and G, which are detected by the X-ray scattering scattering measurement, the ortho-rhombic lamellar crystalline structure of the product F, and the hexagonal lamellar crystalline structure of the product G, respectively, is present on the surface of the treated stratum corneum.

On the surface of the stratum corneum treated with the product F, the X-ray angle scattering measurement has two sharp peaks at 4.16 Å and 3.71 Å, which is characteristic of the ortho-rhombic lamellar crystalline structure of the product F, while the X-ray angle scattering measurement of the surface of the stratum corneum treated with the product G showed only a single peak at 4.16 Å, which is characteristic of the hexagonal lamellar crystalline structure of the conventional product G.

In deeper layers of the stratum corneum, however, it can be demonstrated that this peak disappears at 4.16 Å of the stratum corneum treated with the product G, while in deeper layers of the stratum corneum treated with the product F according to the invention, both peaks are at 4.16 Å and 3.71 Å.

From this it can be concluded that the crystallinity of the conventional product G is completely lost in the deeper layers of the stratum corneum, which leads to a loss of the barrier-supporting function, while the product F according to the invention retains its crystallinity even in deeper layers of the stratum corneum, and thus the barrier-supporting function required and desired for skin protection and skin care acts not only on the surface but also in the depth of the stratum corneum.

What is claimed is:

1. A cosmetic product which contains, in addition to water, at least one hydrogenated phospholipid in a concentration of at least 0.7% by weight, at least one divalent and/or trivalent alcohol and at least one wax, wherein
a) in the cosmetic product, a weight ratio of hydrogenated phospholipid to the wax varies between 1:0.3 and 1:1.5,
b) in the cosmetic product, the hydrogenated phospholipid is at least partially present in an ortho-rhombic lamellar crystalline structure, and
c) the wax is incorporated in the ortho-rhombic lamellar crystalline structure and/or is attached to the ortho-rhombic lamellar crystalline structure.

2. The cosmetic product according to claim 1, wherein the at least one hydrogenated phospholipid is selected from the group comprising hydrogenated phosphatidylethanolamine, hydrogenated phosphatidylinositol, hydrogenated phosphatidylcholine, hydrogenated lyso-phosphatidylcholine, hydrogenated phosphatidylserine, and hydrogenated phosphatidic acid.

3. The cosmetic product according to claim 1, wherein the hydrogenated phospholipid is a hydrogenated phosphatidylcholine and has a concentration of hydrogenated phosphatidylcholine of at least 60% by weight.

4. The cosmetic product according to claim 1, wherein the wax is a vegetable wax and is isolated from leaves, needles, stalks, roots, rinds, bran, peels, seeds, flowers and/or fruits.

5. The cosmetic product according to claim 4, wherein the vegetable wax is selected from the group comprising carnaubal wax, candelilla wax, ouricuri wax, sugar cane wax, retamow wax, caranday wax, raffia wax, columbine wax, esparto wax, alfalfa wax, bamboo wax, hemp wax, Douglas Fir wax, cork wax, sisal wax, flax wax, cotton wax, dammar wax, cereal wax, tea wax, coffee wax, ocatilla wax, citrus aurantium dulcis peel wax, ficus cerifer wax, orange wax, sunflower seed wax, sunflower seed shell wax, Brussels sprout wax, tobacco plant wax, pumpkin seed wax, corn wax, prickly cactus wax and oleander wax.

6. The cosmetic product according to claim 1, wherein the wax is carnauba wax, sunflower seed wax, rice bran wax, rice wax or a mixture of these waxes.

7. The cosmetic product according to claim 6, wherein the carnauba wax, sunflower seed wax, rice wax and/or the rice bran wax contain saturated C₂₂-C₂₆ fatty acids as main fatty acid components.

8. The cosmetic product according to claim 7, wherein the cosmetic product comprises the carnauba wax, sunflower seed wax, rice wax and/or rice bran wax, in which the main fatty acid component of the respective wax is present in a concentration between 15% by weight and 33% by weight, based on the weight of the respective wax.

9. The cosmetic product according to claim 8, wherein the cosmetic product comprises the carnauba wax, sunflower seed wax, rice wax and/or rice bran wax, in which the main fatty acid component of the respective wax is present in a concentration between 23% by weight and 28% by weight, based on the weight of the respective wax.

10. The cosmetic product according to claim 7, wherein the carnauba wax, sunflower seed wax, rice wax and/or the rice bran wax, contains free C₁₆-C₂₄ fatty acids.

11. The cosmetic product according to claim 10, wherein the carnauba wax, sunflower seed wax, rice wax and/or rice bran wax, contains the free C₁₆-C₂₄ fatty acids in a concentration of between 1% by weight and 1.8% by weight, based on the weight of the respective wax.

12. The cosmetic product according to claim 11, wherein the concentration of the unsaturated fatty acids in the free fatty acids varies between 0.05% by weight and 0.4% by weight, based on the weight of the respective wax.

13. The cosmetic product according to claim 1, wherein the cosmetic product contains a diol and/or glycerin as the at least one dihydric or trivalent alcohol.

14. The cosmetic product according to claim 1, wherein the cosmetic product contains, as an alcohol, glycerin, pentylene glycol, 1,2-pentanediol, 1,2-hexanediol, octanediol and/or butylcyclohexanol.

15. The cosmetic product according to claim 1, wherein the cosmetic product contains isostearyl isostearate as a further ingredient in a concentration between 0.5% by weight and 35% by weight.

16. The cosmetic product according to claim 1, wherein the cosmetic product contains a concentration of hydrogenated phosphatidylcholine between 0.7% by weight and 8% by weight.

17. The cosmetic product according to claim 1, wherein the cosmetic product further has a UV filter, a skin-protecting active ingredient, a skin-care active ingredient, a smoothing active ingredient, a skin-softening active ingredient, a skin-whitening active ingredient, a tanning active ingredient, a deodorizing active ingredient, a depilatory active ingredient, a moisturizing active ingredient, an active ingredient for the care and treatment of hypersensitive skin, an active ingredient for the treatment and care of infected, irritated or diseased skin, an active ingredient for prophylaxis against insect bites, a greasing active ingredient, an anti-inflammatory active ingredient and/or a moisturizing active ingredient.

18. The cosmetic product according to claim 1, wherein the cosmetic product has such an active substance selected from the group comprising butylcyclohexanol, resolvin, farnesyl pyrophosphates, capsazepines, cinnamides, carboxamides and palmitoyl tripeptides-8.

19. The cosmetic product according to claim 18, wherein the cosmetic product contains butylcyclohexanol, which is at least one tertiary butylcyclohexanol.

20. The cosmetic product according to claim 18, wherein the cosmetic product has a mixture of the butylcyclohexanol and pentylene glycol.

21. The cosmetic product according to claim 18, wherein a concentration of the above-mentioned active ingredients in the cosmetic product varies between 0.01% by weight and 2.5% by weight.

22. A concentrate for the manufacture of the cosmetic product according to claim 1, wherein
a) the concentrate is prepared by dilution with an aqueous system,
b) wherein the concentrate contains at least one hydrogenated phospholipid, water, at least one dihydric and/or trihydric alcohol and at least one vegetable wax,
c) wherein the concentrate can be diluted with the aqueous system with the formation of the cosmetic product in a volume ratio between 1:0.3 and 1:15,
d) the concentrate has such a concentration of hydrogenated phospholipid that, depending on the desired dilution, the cosmetic product produced therefrom by dilution contains at least 0.7% by weight of the hydrogenated phospholipid,
e) wherein in the concentrate, the weight ratio of hydrogenated phospholipid to the vegetable wax varies between 1:0.3 and 1:1.5,
f) wherein in the concentrate, the hydrogenated phospholipid is at least partially present in an ortho-rhombic lamellar structure, and
g) wherein in the concentrate, the wax is incorporated in the ortho-rhombic lamellar crystalline structure and/or is attached to the ortho-rhombic lamellar crystalline structure.

23. A concentrate according to claim 22, wherein the aqueous system has at least one cosmetic active ingredient.

24. (canceled)
